(19) United States
(12) Patent Application Publication
(10) Pub. No.: US 2003/0069169 A1
(43) Pub. Date: Apr. 10, 2003

(54) Co-administration of melanocortin receptor agonist and phosphodiesterase inhibitor for treatment of cyclic-AMP associated disorders

(76) Inventors: John E. Macor, Guilford, CT (US); Kenneth E. Carlson, West Windsor, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/090,258

(22) Filed: Mar. 4, 2002

Related U.S. Application Data
(60) Provisional application No. 60/273,206, filed on Mar. 2, 2001.
(60) Provisional application No. 60/273,291, filed on Mar. 2, 2001.
(60) Provisional application No. 60/289,719, filed on May 9, 2001.

(51) Int. Cl. A61K 38/00

(52) U.S. Cl. 514/2

(57) ABSTRACT

Co-administration of a melanocortin receptor agonist, particularly an MC-1R or MC-4R agonist, and a cAMP phosphodiesterase inhibitor is described for modulating levels of cyclic adenoise 3′,5′ monophosphate (cAMP) in a mammal. The inventive co-administration is useful in the treatment of diseases affected by activity of cAMP-PDE, including without limitation, inflammatory bowel disease, irritable bowel syndrome, rheumatoid arthritis, osteoarthritis, pancreatis, psoriasis, migraine, Alzheimer's Disease, Parkinson's disease, transplant rejection, asthma, acute respiratory distress syndrome, chronic obstructive pulmonary disease, stroke, and neurodegeneration of, and consequences of traumatic brain injury.

### RELATED INVENTIONS

This application claims the benefit of priority of U.S. applications Serial Nos. 60/273,206, and 60/273,291, filed Mar. 2, 2001, and U.S. Serial No. 60/289,719 filed May 9, 2001, the entire contents of which are incorporated herein by reference.

### FIELD OF THE INVENTION

The present invention relates to methods for treating diseases associated with intracellular levels of cAMP comprising co-administration of at least one compound that is a melanocortin receptor agonist and at least one compound that is a cAMP-PDE inhibitor, and to pharmaceutical compositions for practicing the claimed methods.

### BACKGROUND OF THE INVENTION

Cyclic adenoise 3′,5′ monophosphate (cyclic AMP or cAMP) is a nucleotide messenger associated with inflammatory cell activity; it mediates the functional responses of cells to a multitide of hormones and neurotransmitters, including NF-κB. NF-κB is a pivotal component of the pro-inflammatory cascade, and its activation is a central event in initiating many inflammatory diseases. In a typical inflammatory response, NF-κB is activated in response to an inflammatory stimulus and once activated, induces expression of a wide array of pro-inflammatory genes.

cAMP is hydrolyzed to the inactive 5′ nucelotide adenoisine monophosphate (AMP), by certain phosphodiesterases (PDEs). PDEs comprise a group of enzymes that hydrolyze the phosphodiester bond of cyclic nucleotides to form inactive nucleotides, e.g., certain PDEs hydrolyze cAMP to AMP and certain PDEs hydrolyze cyclic 3′,5′-guanosine monophosphate (cGMP) to the inactive 5′ nucleotide guanosine monophosphate (GMP). At least eleven families of PDEs are now known to exist, which are grouped according to their specificity toward hydrolysis of cAMP or cGMP, their sensitivity to calcium regulation, and/or their selective inhibition by various compounds. For example, type 5, 6 and 9 PDEs modulate cGMP content only and do not hydrolyze cAMP. PDEs type 3, 4, 7 and 8 are specific for cAMP, and other PDEs (types 1, 2, 10 and 11) have dual specificity.

Since cAMP is associated with inflammatory cell activity, it was believed that inhibition of those PDEs that degrade cAMP could provide a therapeutic benefit in treating inflammatory disease. PDE inhibitors have been extensively studied as therapeutic targets in treating inflammatory disease, particularly inflammatory respiratory diseases such as asthma, chronic obstructive pulmonary disease (COPD), and acute respiratory distress syndrome (ARDS). However, challenges have been encountered in developing therapeutically-effective PDE inhibitors. PDE inhibitors have a relatively modest therapeutic effect, and because PDEs play an important role in many cellular interactions, non-specific PDE inhibition has been associated with significant adverse side effects.

Melanocortin peptides, particularly α-melanocyte stimulating hormone ((α-MSH), have a wide range of effects on biological functions including feeding behavior, pigmentation, and exocrine function. The biological effects of α-MSH are mediated by a sub-family of G protein-coupled receptors, termed melanocortin receptors. There are four melanocortin receptors: MC-1R, MC-3R, MC-4R, and MC-5R (MC-2R is not a receptor for (α-MSH but is the adrenocorticotropic hormone {ACTH} receptor).

MC-1R is an important regulator of melanin production and coat color in animals (skin color in humans). Recently, evidence has shown that α-MSH induces a potent anti-inflammatory effect in both acute and chronic models of inflammatory disease. The anti-inflammatory actions of α-MSH are likely mediated by MC-1R. MC-1R is expressed in cells that are important regulators of the immune response: monocyte/macrophages, neutrophils, endothelial, and mast cells. Stimulation with α-MSH results in a dampening of the inflammatory response in these cells. MC-3R, MC-4R and MC-5R are implicated in feeding behavior, body weight, and exocrine gland function. Much attention has been focused on the study of MC-3R and MC-4R modulators and their use in treating sexual dysfunction and body weight disorders such as obesity and anorexia. WO 00/53148 discloses methods of treating erectile dysfunction using MC-4R agonists and cGMP inhibitors. See also International publication WO 00/74679, which claims compositions that comprise a combination of an MC-4R agonist and a type 5 cGMP PDE inhibitor.

The present invention provides methods for treating conditions associated with intracellular levels of cAMP comprising co-administration of at least one compound that is an MC-1R agonist and at least one compound that is a cAMP-phosphopdiesterase (PDE) inhibitor, as well as methods for treating such conditions with at least one compound that is an MC-4R agonist and at least one compound that is a cAMP-PDE inhibitor. To the inventor's knowledge, the administration of an MC-1R or MC-4R agonist and cAMP-PDE inhibitor has not been described for treating inflammatory and immune diseases. U.S. Pat. No. 6,060,051 issued May 9, 2000, describes methods for treating multiple sclerosis comprising administration of synergistically effective amounts of a PDE 4 compound and an anti-inflammatory agent, particularly an interferon.

### SUMMARY OF THE INVENTION

The instant invention is based on the discovery that co-administration of a compound that is an agonist of MC-1R and a compound that is an inhibitor of cAMP-PDE produces enhanced therapeutic benefits in treating cAMP-associated conditions. According to one aspect of the invention, there is provided a method for regulating cyclic adenoise 3′,5′ monophosphate (cAMP) production in a mammal comprising administering to the mammal a combination of (i) an amount of at least one compound that is an effective agonist of MC-1R (preferably a selective MC-1R agonist) and (ii) an amount of at least one compound that is an inhibitor of cAMP-PDE. According to another aspect of the invention, there is provided a method for regulating cAMP production in a mammal comprising administering to the mammal a combination of (i) an amount of at least one compound that is an effective agonist of MC-4R (preferably a selective MC-4R agonist) and (ii) an amount of at least one compound that is an inhibitor of cAMP-PDE. With the invention, the combination may comprise use of a subtherapeutically-effective amount of the melanocortin-receptor agonist and/or use of a subtherapeutically-effective amount of the cAMP-PDE inhibitor; however, because the melanocortin-receptor agonist and cAMP-PDE inhibitor work together to modulate cAMP levels, a therapeutically-effective regulation of cAMP levels is achieved with the inventive combination.

The melanocortin receptor agonist of the inventive methods and compositions may include any compound having activity in agonizing MC-1R as defined herein, and/or any compound having activity in agonizing MC-4R as defined herein. Preferred melanocortin receptor agonists are novel compounds described in U.S. patent applications Ser. Nos. 60/273,206 and 60/273,291, both filed Mar. 2, 2001, having common inventor(s) and the same assignee herein, and in the corresponding non-provisional patent applications Ser. Nos.______,filed Mar. 4, 2002, incorporated herein. To the inventor's knowledge, small molecule compounds useful as MC-1R agonists had not been previously described, although WO 99/57148 to WA Pharma AB (1999), “Melanocortin 1 Receptor Selective Compounds,” and WO 99/43709 to The Regents of the Univ. of Calif., “Melanocortin Receptor Antagonists and Modulations of Melanocortin Receptor Activity,” disclose large polypeptides reportedly having activity as MC-1R modulators. Compounds that reportedly are agonists of MC-4R are disclosed in WO 00/74679, WO 01/70708, WO 01/91752, and WO 02/00654, incorporated herein by reference, and such compounds may be useful in the inventive methods claimed herein.

The PDE inhibitor of the inventive methods and compositions may comprise any compound having activity as a cAMP-PDE inhibitor. Thus, inhibitors of PDEs type 1, 2, 3, 4, 7, 8, 10 and 11 may be used according to the invention. An advantage of this invention is that the cAMP-PDE inhibitor need not comprise a selective PDE type 4 inhibitor or an inhibitor having selectivity for one particular type of PDE 4 isoenzyme. The PDE inhibitor may comprise at least one compound of formula (IIa) (rolipram); formula (IIb) (denbutyline); formula (IIc) (theophylline, i.e., 1,2-dimethylxanthine); formula (IId) (XT-44), and/or formula (Ie) (ARIFLO™ i.e., cis-4-cyano-4-[3-(cyclopentyloxy)-4-methoxyphenyl]cyclohexane-1-carboxylic acid); and/or pharmaceutically-acceptable salts or derivatives thereof:

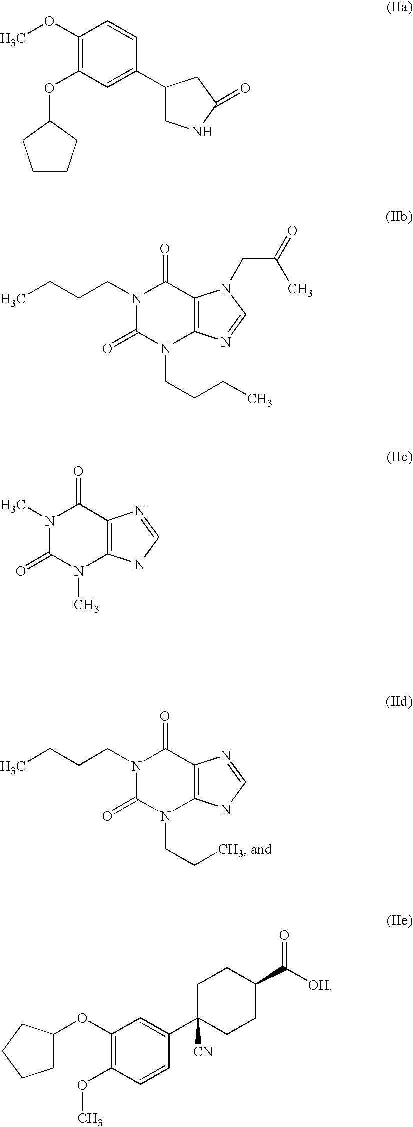

Other exemplary cAMP-PDE inhibitors including PDE type 4 and/or 7 inhibitors that may be used according to the invention are described hereinafter.

### BRIEF DESCRIPTION OF THE DRAWINGS

FIG. 1 is a bar graph showing the results of in vivo administration of a selective MC-1R agonist according to formula (I) on LPS-induced TNF-α production in mice; and

FIG. 2 is a bar graph showing the results of in vivo administration of a melanocortin receptor agonist alone, a cAMP-PDE inhibitor (i.e., rolipram) alone, and the melanocortin receptor agonist in combination with the cAMP-PDE inhibitor, on LPS-induced TNF-α production in mice.

### DETAILED DESCRIPTION OF THE INVENTION

The instant invention is based on the discovery that small molecule compounds that are agonists of MC-1R are effective as anti-inflammatory, immunosuppressive, skin pigmentation, cardiovascular, and neurogenerative agents. Additionally, small molecule compounds have been discovered that are agonists of MC-4R and effective for treating bodyweight, neurodegenerative, and other disorders associated with the activity of MC-4R. The melanocortin-receptor agonists elevate intracellular levels of cAMP. However, elevated intracellular levels of cAMP upon administration of an MC-1R agonist (or MC-4R agonist) may cause the cells to express enhanced levels of PDE enzymes that hydrolyze the phosphodiester bond of cAMP. PDEs are efficient hydrolyzing enzymes. Thus, an overactive PDE response can reduce the therapeutic benefits to be achieved when a melanocortin receptor agonist is administered to a patient.

With the methods and compositions of this invention, an amount of at least one melanocortin receptor agonist (selected from MC-1R and MC-4R agonist), elevates intracellular levels of cAMP, and an amount of at least one cAMP-PDE inhibitor blocks degradation of cAMP to provide an enhanced therapeutic effect in treating a cAMP-associated condition as compared with use of the MC-1R agonist, MC-4R agonist, or cAMP-PDE inhibitor alone. The invention provides the advantage of promoting effective modulation of cAMP levels with administration of a melanocortin receptor agonist, as the inventive combination blocks or mitigates an adverse PDE response to melanocortin receptor activation.

A further advantage provided by the instant invention is that the combination of melanocortin-receptor agonist and cAMP-PDE inhibitor allows for administration of a reduced dose of the cAMP-PDE inhibitor and/or the melanocortin-receptor agonist while achieving the same degree of cAMP elevation that would be achieved upon administering a larger dose of a cAMP-PDE inhibitor or melanocortin-receptor agonist, when administered alone. Thus, with the invention, the same or similar therapeutic benefits can be achieved as with a therapeutically-effective dose of a cAMP-PDE inhibitor, while avoiding the use of a therapeutically-effective dose of cAMP-PDE inhibitor and the adverse side effects associated therewith.

The following are definitions of terms used in this specification. The initial definition provided for a group or term herein applies to that group or term throughout the present specification, individually or as part of another group, unless otherwise indicated.

The term “therapeutically-effective amount” is intended to refer to the amount of compound or composition that is needed to achieve a desired therapeutic effect in treating at least one cAMP-associated condition in a mammal.

The term “subtherapeutically-effective amount” when used herein with reference to an MC-1R agonist, MC-4R agonist, or a cAMP-PDE inhibitor means that the amount of the compound or composition is not, by itself, effective to achieve the desired therapeutic effect for the condition being treated.

The term “additive effect” as used herein means that, when two or more compounds are administered in combination, at least one effect is greater than would be achieved when one of the compounds is administered alone as an individual single agent. A “maximum additive effect” means that when two or more compounds are administered in combination, the overall effect is the same as compared to when the two compounds are administered alone as individual single agents and the effects added.

The term “synergistically-effective result” or “synergistically-effective” as used herein means that, when two or more compounds are administered in combination, at least one effect is greater than would be achieved when the two or more compounds are administered alone as individual single agents and the effects added. In other words, a “synergistic-effect” means any degree of effect that is greater than the maximum additive effect.

The term “effect” when used with reference to additive effects and synergistic effects may be an anti-inflammatory effect, an anti-thrombotic effect, a reduction in side effects or pain effect, or any other desired therapeutic or phrophylaxis effect.

The terms “co-administration,” “in combination with,” “administered in combination,” and the like, when used herein are meant to refer to use of both a melanocortin-receptor agonist (MC-1R or MC-4R), and cAMP-PDE inhibitor to treat a cAMP-associated condition. The combined use of the MC-1R agonist and cAMP-PDE inhibitor may be performed simultaneously or sequentially in any order. With the invention, the compounds may be combined in one pharmaceutically-acceptable carrier, or they may be placed in separate carriers and administered to the patient at different times. Each of these situations is contemplated as falling within the meaning of “co-administration” or “combination,” the important consideration being that the compounds should be administered sufficiently close in time that there is at least some temporal overlap in the biological effects generated by the compounds on the mammal being treated.

The term “MC1R agonist” means a compound that has demonstrated activity in agonizing the MC1R. A “selective MC-1R agonist” means a compound that has greater activity in agonizing MC-1R than any other melanocortin receptor. The selective MC-1R agonist may have some, albeit lesser, activity in agonizing or antagonizing MC-3R, MC-4R, and/or MC-5R. For example, a “moderately selective MC1R agonist” means a compound that is about 100-fold less potent at MC-3R, MC-4R and/or MC-5R than at MC-1R, and a “highly selective MC1R agonist” means a compound that is more than 1000-fold less potent at MC-3R, MC-4R and/or MC-5R than at MC-1R. A compound falling within these ranges (about 100 to 1000-fold less potent at MC-3R, MC-4R and/or MC-5R than at MC-1R) is thus moderately to highly selective, as those terms are used herein.

The term “MC4R agonist” means a compound that has demonstrated activity in agonizing the MC4R. A “selective MC-4R agonist” means a compound that has greater activity in agonizing MC-4R than any other melanocortin receptor. The selective MC-4R agonist may have some, albeit lesser, activity in agonizing or antagonizing MC-1R, MC-3R, and/or MC-5R. For example, a “moderately selective MC4R agonist” means a compound that is about 100-fold less potent at MC-1R, MC-3R and/or MC-5R than at MC-4R, and a “highly selective MC4R agonist” means a compound that is more than 1000-fold less potent at MC-1R, MC-3R and/or MC-5R than at MC-4R. A compound falling within these ranges (about 100 to 1000-fold less potent at MC-1R, MC-3R and/or MC-5R than at MC-4R) is thus moderately to highly selective, as those terms are used herein.

The term “cAMP-PDE inihibitor” means a compound that inhibits PDEs that hydrolyze cAMP. Thus, for example, a cAMP-PDE inhibitor does not include a PDE type 5 inhibitor, as type 5 PDEs hydrolyze only cGMP, not cAMP. However, type 1, 2, 10 and 11 PDEs hydrolyze both cAMPs and cGMPs, and thus inhibitors of those PDEs are cAMP-PDE inhibitors. The term “selective cAMP-PDE inhibitor” means a compound that has greater activity in inhibiting those PDEs that hydrolyze cyclic AMP as compared with cyclic GMP. The selective cAMP-PDE inhibitor may have some, albeit lesser, activity in inhibiting PDEs that hydrolyze cGMP (e.g. in the case of PDEs type 1, 2, 10 and 11). PDE type 3, 4, 7 and 8 inhibitors are necessarily selective cAMP-PDE inhibitors as the term is used herein as they are specific for cAMP.

The term “alkyl” refers to straight or branched chain hydrocarbon groups having 1 to 12 carbon atoms, preferably 1 to 8 carbon atoms. Lower alkyl groups, that is, alkyl groups of 1 to 4 carbon atoms, are most preferred. When a subscript is used with reference to an alkyl or other group, the subscript refers to the number of carbon atoms that the group may contain.

The term “substituted alkyl” refers to an alkyl group as defined above having one, two or three substituents selected from the group consisting of halo, amino, cyano, keto (═O), —ORₐ, —SRₐ, NRₐR_{b}, —(C═O)Rₐ, —CO₂Rₐ, —(C═O)NRₐR_{b}, —NRₐC(═O)R_{b}, NRₐCO₂R_{b}, —OC(═O)Rₐ, —OC(═O)NRₐR_{b}, —NR_{c}C(═O)NRₐR_{b}, NRₐSO₂R_{d}, SO₂R_{d}, SO₃R_{d}, cycloalkyl, aryl, heteroaryl, or heterocycle, wherein the groups Rₐ, R_{b}, and R_{c}, are selected from hydrogen, C₁₋₆alkyl, aryl, heteroaryl, heterocycle, cycloalkyl, or C₁₋₆alkyl substituted with halogen, hydroxy, methoxy, nitro, amino, cyano, —(C═O)H, —CO₂H, —(C═O)alkyl, —CO₂alkyl, —NH(alkyl), —NH(cycloalkyl), —N(alkyl)₂, carboxy, acyl, —C(═O)H, —C(═O)phenyl, —CO₂-alkyl, cycloalkyl, —(C═O)NH₂, —(C═O)NH(alkyl), —(C═O)NH(cycloalkyl), —(C═O)N(alkyl)₂, —C(═O)—(CH₂)₁₋₂NH₂, —C(═O)—(CH₂)₁₋₂NH(alkyl), —C(═O)—(CH₂)₁₋₂N(alkyl)₂, —NH—CH₂-carboxy, —NH—CH₂—CO₂-alkyl, phenyl, benzyl, phenylethyl, or phenyloxy. The group R_{d} may be selected from the same groups as Rₐ, R_{b} and R_{c} but is not hydrogen. Alternatively, the groups Rₐ and R_{b} may together form a heterocyclo or heteroaryl ring. It should be understood that when a substituted alkyl group is substituted with an aryl, cycloalkyl, heteroaryl, or heterocyclo, such rings are as defined below and thus may have one to three substituents as set forth below in the defintions for these terms.

When the term “alkyl” is used as a suffix following another specifically named group, e.g., arylalkyl, heteroarylalkyl, the term defines with more specificity at least one of the substituents that the substituted alkyl will contain. For example, arylalkyl refers to an aryl bonded through an alkyl, or in other words, a substituted alkyl group having from 1 to 12 carbon atoms and at least one substituent that is aryl (e.g., benzyl or biphenyl). “Lower arylalkyl” refers to substituted alkyl groups having 1 to 4 carbon atoms and at least one aryl substituent.

The term “alkenyl” refers to straight or branched chain hydrocarbon groups having 2 to 12 carbon atoms and at least one double bond. Alkenyl groups of 2 to 6 carbon atoms and having one double bond are most preferred.

The term “alkynyl” refers to straight or branched chain hydrocarbon groups having 2 to 12 carbon atoms and at least one triple bond. Alkynyl groups of 2 to 6 carbon atoms and having one triple bond are most preferred. A substituted alkenyl or alkynyl will contain one, two, or three substituents as defined above for alkyl groups.

The term “alkylene” refers to bivalent straight or branched chain hydrocarbon groups having 1 to 12 carbon atoms, preferably 1 to 8 carbon atoms, e.g., {—CH₂—}ₙ, wherein n is 1 to 12, preferably 1-8. Lower alkylene groups, that is, alkylene groups of 1 to 4 carbon atoms, are most preferred. The terms “alkenylene” and “alkynylene” refer to bivalent radicals of alkenyl and alknyl groups, respectively, as defined above. Substituted alkylene, alkenylene, and alkynylene groups may have substituents as defined above for substituted alkyl groups.

The term “alkoxy” refers to the group ORₑ wherein Rₑ is alkyl, substituted alkyl, alkenyl, substituted alkenyl, alkynyl, substituted alkynyl, heterocycle, or cycloalkyl. Thus, an alkoxy includes such groups as methoxy, ethoxy, cyclopropyloxy, pyrrolidinyloxy, and so forth. The term “aryloxy” refers to the groups O(aryl) or O(heteraryl), wherein aryl and heteroaryl are as defined below.

The term “alkylthio” refers to an alkyl or substituted alkyl group as defined above bonded through one or more sulfur (—S—) atoms, e.g., —S (alkyl) or —S (alkyl-Rₐ).

The term “alkylamino” refers to an alkyl or substituted alkyl group as defined above bonded through one or more nitrogen (—NR_{f}—) groups, wherein R_{f} is hydrogen, alkyl, substituted alkyl, or cycloalkyl.

The term “acyl” refers to an alkyl or substituted alkyl group as defined above bonded through one or more carbonyl {—C(═O)—} groups. When the term acyl is used in conjunction with another group, as in acylamino, this refers to the carbonyl group {—C(═O)} linked to the second named group. Thus, acylamino refers to —C(═O)NH₂, substituted acylamino refers to the group —C(═O)NRR, and acylaryl refers to —C(═O)(aryl).

The term “aminoacyl” refers to the group —NR_{f}C(═O)R_{g}, wherein R_{g} is hydrogen, alkyl, or substituted alkyl, and R_{f} is as defined above for alkylamino groups.

The term “halo” or “halogen” refers to chloro, bromo, fluoro and iodo.

The term “carboxy” when used alone refers to the group CO₂H. Carboxyalkyl refers to the group CO₂R, wherein R is alkyl or substituted alkyl.

The term “sulphonyl” refers to a sulphoxide group (i.e., —S(O)₁₋₂—) linked to an organic radical including an alkyl, alkenyl, alkynyl, substituted alkyl, substituted alkenyl, or substituted alkynyl group, as defined above. The organic radical to which the sulphoxide group is attached may be monovalent (e.g., —SO₂-alkyl), or bivalent (e.g., —SO₂-alkylene, etc.)

The term “amidino” refers to the group

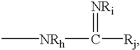

and the term “guanidino” refers to the group

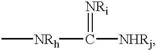

wherein for each of amidino and guanidino Rₕ, Rᵢ, and Rⱼ may be hydrogen, alkyl, or substituted alkyl, or any two of Rₕ, Rᵢ, and Rⱼ may join to form a heterocyclo or heteroaryl ring with the other of Rₕ, Rᵢ, and Rⱼ comprising hydrogen, alkyl, or substituted alkyl.

The term “cycloalkyl” refers to substituted and unsubstituted monocyclic or bicyclic hydrocarbon groups of 3 to 9 carbon atoms which are, respectively, fully saturated or partially unsaturated, including a fused aryl ring, for example, an indan. A cycloalkyl group may be substituted by one or more (such as one to three) substituents selected from alkyl, substituted alkyl, aminoalkyl, halogen, cyano, nitro, trifluoromethyl, hydroxy, alkoxy, alkylamino, sulphonyl, —SO₂(aryl), —CO₂H, —CO₂-alkyl, —C(═O)H, keto, —C(═O)—(CH₂)₁₋₂NH₂, —C(═O)—(CH₂)₁₋₂NH(alkyl), —C(═O)—(CH₂)₁₋₂N(alkyl)₂, acyl, aryl, heterocylcle, heteroaryl, or another cycloalkyl ring of 3 to 7 carbon atoms. The term “cycloalkylene” refers to a cycloalkyl forming a link or spacer between two other groups, i.e., a cycloalkylene is a cycloalkyl that is bonded to at least two other groups. The term cycloalkyl includes saturated or partially unsaturated carbocyclic rings having a carbon-carbon bridge of three to four carbon atoms or having a benzene ring joined thereto. When the cycloalkyl group is substituted with a further ring, said further ring may have one to two substituents selected from Rₖ, wherein Rₖ is lower alkyl, hydroxy, lower alkoxy, amino, halogen, cyano, trifluoromethyl, trifluoromethoxy, nitro, and lower alkyl substituted with one to two hydroxy, lower alkoxy, amino, halogen, cyano, trifluoromethyl, trifluoromethoxy, and/or nitro.

The term “aryl” refers to substituted and unsubstituted phenyl, 1-naphthyl and 2-naphthyl, with phenyl being preferred. The aryl may have zero, one, two or three substituents selected from the group consisting of alkyl, substituted alkyl, alkoxy, alkylthio, halo, hydroxy, nitro, cyano, amino, trifluoromethyl, trifluoromethoxy, sulphonyl, —SO₂(aryl), —NH(alkyl), —NH(cycloalkyl), —N(alkyl)₂, carboxy, acyl, —C(═O)H, —C(═O)phenyl, —CO₂-alkyl, cycloalkyl, —(C═O)NH₂, —(C═O)NH(alkyl),—(C═O)NH(cycloalkyl), —(C═O)N(alkyl)₂, —NH—CH₂-carboxy, —NH—CH₂—CO₂-alkyl, —C(═O)—(CH₂)₁₋₂NH₂, —C(═O)—(CH₂)₁₋₂NH(alkyl), —C(═O)—(CH₂)₁₋₂N(alkyl)₂, phenyl, benzyl, phenylethyl, phenyloxy, phenylthio, heterocyclo, heteroaryl, or a C₃₋₇cycloalkyl ring. The term “arylene” refers to an aryl as defined above forming a link or spacer between two other groups, i.e., an arylene is an aryl that is bonded to at least two other groups. When the aryl group is substituted with a further ring, said further ring may have one to two substituents selected from Rₖ, wherein Rₖ is defined as above.

The term “carbocyclo” or “carbocyclic” refers to a cyclic group in which all ring atoms are carbon, including optionally-substituted cycloalkyl and aryl groups, as defined herein.

The term “heterocyclo” or “heterocycle” refers to substituted and unsubstituted non-aromatic 3 to 7 membered monocyclic groups, 7 to 11 membered bicyclic groups, and 10 to 15 membered tricyclic groups which have at least one heteroatom (O, S or N) in at least one of the rings. Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. The heterocyclo group may be attached at any available nitrogen or carbon atom. The heterocyclo ring may contain one, two or three substituents selected from the group consisting of halo, amino, cyano, alkyl, substituted alkyl, trifluoromethyl, trifluoromethoxy, sulphonyl, —SO₂(aryl), —NH(alkyl), —NH(cycloalkyl), —N (alkyl)₂, alkoxy, alkylthio, hydroxy, nitro, phenyl, benzyl, phenylethyl, phenyloxy, phenylthio, carboxy, —CO₂-alkyl, cycloalkyl, —C(═O)H, acyl, —(C═O)NH₂, —(C═O) NH(alkyl), —(C═O)NH(cycloalkyl), —(C═O) N(alkyl)₂, —NH—CH₂-carboxy, —NH—CH₂—CO₂-alkyl, —C(═O)—(CH₂)₁₋₂NH₂, —C(═O)—(CH₂)₁₋₂NH(alkyl), —C(═O)—(CH₂)₁₋₂N(alkyl)₂, heterocyclo, heteroaryl, a C₃₋₇cycloalkyl ring. keto, ═N—OH, ═N—O-lower alkyl, or a five or six membered ketal, i.e., 1,3-dioxolane or 1,3-dioxane. The heterocyclo ring may have a sulfur heteroatom that is substituted with one or more oxygen (═O) atoms, as for example, in

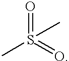

The term “heterocyclene” refers to a heterocycle as defined above forming a link or spacer between two other groups. When the heterocyclo group is substituted with a further ring, said further ring may have one to two substituents selected from Rₖ, wherein Rₖ is defined as above.

Exemplary monocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl.

The term “heteroaryl” refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain one, two or three substituents selected from the group consisting of halo, amino, cyano, alkyl, substituted alkyl, trifluoromethyl, trifluoromethoxy, sulphonyl, —SO₂(aryl), —NH(alkyl), —NH(cycloalkyl), —N(alkyl)₂, alkoxy, alkylthio, hydroxy, nitro, phenyl, benzyl, phenylethyl, phenyloxy, phenylthio, carboxy, —CO₂-alkyl, cycloalkyl, —C(═O)H, acyl, —(C═O)NH₂, —(C═O)NH(alkyl), —(C═O)NH(cycloalkyl), —(C═O) N(alkyl)₂, —NH—CH₂-carboxy, —NH—CH₂—CO₂-alkyl, —C(═O)—(CH₂)₁₋₂NH₂, —C(═O)—(CH₂)₁₋₂NH(alkyl), —C(═O)—(CH₂)₁₋₂N(alkyl)₂, heterocylco, heteroaryl, or a C₃₋₇cycloalkyl ring. The heterocyclo ring may have a sulfur heteroatom that is substituted with one or more oxygen (═O) atoms, as for example, in

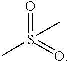

The term “heteroarylene” or “heterarylene” refers to a heteroaryl as defined above forming a link or spacer between two other groups, i.e., it is a heteroaryl that is bonded to at least two other groups. When the heteroaryl group is substituted with a further ring, said further ring may have one to two substituents selected from Rₖ, wherein Rₖ is defined as above.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

When reference is made herein to a particularly-named heterocyclic or heteroaryl group, such as azetidinyl, imidazolyl, piperazinyl, and so forth, the named ring may optionally contain one or more (preferably one to three) substituents selected from the substituents recited above for heteroaryl and heterocyclo groups, as appropriate. The term azetidinyl refers to an optionally-substituted four membered ring having one nitrogen heteroatom, i.e.,

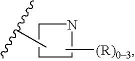

wherein R can be any substituent defined herein for heterocycles and unless otherwise stated, the azetidinyl ring can be attached to another group at any available carbon atom or at the nitrogen atom.

When reference is made to a particularly-named group having at least one heterocyclo, heteroaryl, or carbocyclic ring “joined” thereto, it is meant that two substituents attached to the same, adjacent, or non-adjacent atoms of the particularly-named group may join to form a second or third ring (i.e., the further ring may be fused, bridged or attached in a spiro fashion.) Each ring of these bicyclic or tricyclic groups may be optionally substituted, wherein the substituents are selected from those recited above for cycloalkyl, aryl, heterocyclo and heteroaryl groups. Thus, an imidazole having at least one ring joined thereto may include an aryl-fused imidazole such as benzimidazole having one or more (preferably one to three substituents), to an heteroaryl-fused imidazole such as a pyridoimidazole having one or more (preferably one to three) substituents, and so forth.

Throughout the specification, groups and substituents thereof may be chosen to provide stable moieties and compounds.

The compounds used in the inventive methods and compositions, such as compounds formula I, may form salts and use of such salts is also within the scope of this invention. A reference to a particularly-named MC-1R agonist, MC-4R agonist, or cAMP-PDE inhibitor is understood to include reference to salts thereof, unless otherwise indicated. The term “salt(s)”, as employed herein, denotes acidic and/or basic salts formed with inorganic and/or organic acids and bases. In addition, when an MC-1R agonist, MC-4R agonist, or a cAMP-PDE inhibitor referred to herein contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred.

MC-1R agonists, MC-4R agonists, or cAMP-PDE inhibitors which contain a basic moiety, such as, but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

MC-1R agonists, MC-4R agonists, or cAMP-PDE inhibitors which contain an acidic moiety, such as, but not limited to a carboxylic acid, may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates (preferably hydrates) of the described MC-1R agonists, MC-4R agonists, and cAMP-PDE inhibitors may also be used according to the invention. The term “prodrug”, as employed herein, denotes a compound which, upon administration to a subject, undergoes chemical conversion by metabolic or chemical processes to yield the particularly-claimed MC-1R agonist, MC-4R agonist, or cAMP-PDE inhibitor.

The MC-1R agonists, MC-4R agonists, and cAMP-PDE inhibitors, and salts thereof, may exist in their tautomeric form (for example, as an amide or imino ether). All such tautomeric forms are contemplated herein as part of the present invention.

All stereoisomers of the MC-1R agonists, MC-4R agonists, and cAMP-PDE inhibitors, including enantiomeric forms (which may exist even in the absence of asymmetric carbons) and diastereomeric forms, are contemplated and within the scope of this invention. Individual stereoisomers may, for example, be substantially free of other isomers, or may be admixed, for example, as racemates or with all other or other selected, stereoisomers. The chiral centers of the present invention can have the S or R configuration as defined by the IUPAC 1974 Recommendations.

### Modes of Administration

### Melanocortin-receptor Agonists

The melanocortin-receptor agonist to be used in the inventive combination may comprise a compound of formula (I),

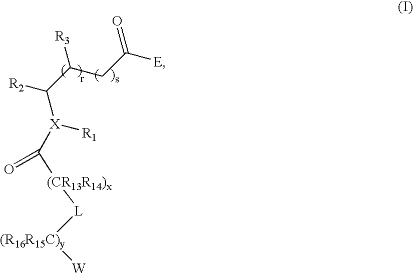

or a pharmaceutically-acceptable salt, hydrate, or prodrug thereof, in which:

L is a bond or —CH(G)—;

X is N or CH;

R₁ is hydrogen or C₁₋₆alkyl or is taken together with R₂ or R₃to form a monocyclic or bicyclic aryl, cycloalkyl, heteroaryl or heterocycle;

R₂ is hydrogen, aryl, cycloalkyl, heteroaryl, or heterocyclo; or C₁₋₆alkyl or C₂₋₆alkenyl optionally substituted with one to three of hydroxy, alkoxy, halogen, cyano, trifluoromethyl, nitro, amino, alkylamino, aryl, cycloalkyl, heteroaryl, and/or heterocyclo; or R₂ is taken together with R₁ or R₃to form a monocyclic or bicyclic aryl, cycloalkyl, heteroaryl or heterocycle;

R₃ is hydrogen or C₁₋₆alkyl or is taken together with R₁ or R₂ to form a monocyclic or bicyclic aryl, cycloalkyl, heteroaryl or heterocycle;

E is E₁, E₂, E₃ or E₄, wherein

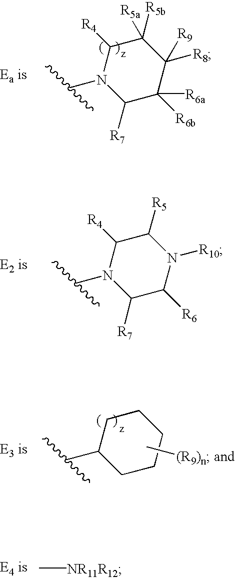

G is selected from C₂₋₆alkenyl, A₃-aryl, —OR₁₈, A₁-heteroaryl, A₁-cyano, A₂ —OR₁₇, A₁-C(═O)R₁₈, A₁-CO₂R₁₈, A₁-C(═O)NR₁₈R₁₉, A₁-OC(═O)R₁₈, A₁-NR₁₈C(═O)R₁₉, A₁-OC(═O)NR₁₈R₁₉, A₁-NR₁₈CO₂R₁₉, A₁-NR₁₈SO₂R₁₇, A₁-SO₂R₁₇, A₁-NR₂₀C(═O)NR₁₈R₁₉, A₁-SR₁₈, A₁-heterocyclo, wherein A₁ is a bond, C₁₋₆alkylene or C₂₋₆alkenylene (straight or branched chain), A₂ is C₁₋₆alkylene or C₂₋₆alkenylene, and A₃ is C₂₋₆alkenylene;

W is selected from —NR₂₁R₂₂, —OR₂₃, —NR₂₁C(═O)R₂₄, —NR₂₁CO₂R₂₄, amidino, guanidino, or a substituted or unsubstituted heterocyclo, heteroaryl, or cycloalkyl selected from azepinyl, azetidinyl, imidazolyl, imidazolidinyl, pyrazolyl, pyridyl, pyrazinyl, pyridazinyl, 1,2-dihydropyridazinyl, pyranyl, tetrahydropyranyl, piperazinyl, homopiperazinyl, pyrrolyl, pyrrolidinyl, piperidinyl, thiazolyl, tetrahydrothiazolyl, thienyl, furyl, tetrahydrofuryl, morpholinyl, isoquinolinyl, tetrahydroisoquinolinyl, tetrazolyl, oxazolyl, tetrahydro-oxazolyl, and C₃₋₇cycloalkyl, wherein said heteroaryl, heterocyclo or cycloalkyl groups may additionally have joined thereto an optionally substituted five-to-seven membered heterocyclic, heteroaryl, or carbocyclic ring;

R₄ and R₇ are independently selected from hydrogen, alkyl, substituted alkyl, halogen, hydroxy, alkoxy, and keto;

R₅, R₅ₐ, R_{5b}, R₆, R₆ₐ, R_{6b}, R₈ and R₉ are independently hydrogen, halogen, cyano, alkyl, substituted alkyl, alkenyl, alkynyl, cycloalkyl, heterocyclo, aryl, heteroaryl, —OR ₂₅, —NR₂₅R₂₆, —SR ₂₅—S(O)ₚR₂₆, —C(═O)R₂₅, —OC(═O) R₂₅, —CO₂R₂₅, —C(═O) NR₂₅R₂₆, —N R₂₅C(═O) R₂₆, —OC(═O) N R₂₅R₂₆, —NR₂₅CO₂R₂₆, —NR₂₇C(═O) NR₂₅R₂₆ or —NR₂₅SO₂R₂₆; or R₅ₐ, and R_{5b}, R₆ₐ and R_{6b}, or R₈ and R₉ taken together form a keto group (═O) or a monocyclic or bicyclic cycloalkyl or heterocyclo joined in a spiro fashion to ring E, or alternatively, R₅ₐ and/or R_{5b} together with R₈ and/or R₉, or R₆ₐ and/or R_{6b} together with R₈ and/or R₉, are taken to form a fused carbocyclic, heterocyclic, or heteroaryl ring;

R₁₀ is selected from hydrogen, alkyl, substituted alkyl, cycloalkyl, aryl, heteroaryl, and hetereocyclo;

R₁₁ is hydrogen or C₁₈alkyl;

R₁₂ is C₁₋₈alkyl, substituted C₁₋₈alkyl, or cycloalkyl;

R₁₃, R₁₄, R₁₅ and R₁₆ are selected independently of each other from hydrogen, alkyl, substituted alkyl, amino, alkylamino, hydroxy, alkoxy, aryl, cycloalkyl, heteroaryl, or heterocyclo, or R₁₃ and R₁₄, or R₁₅ and R₁₆, when attached to the same carbon atom, may join to form a spirocycloalkyl ring;

R₁₇ is alkyl, substituted alkyl, cycloalkyl, aryl, heterocyclo, or heteroaryl;

R₁₈, R₁₉, and R₂₀ are independently selected from hydrogen, alkyl, substituted alkyl, alkenyl, substituted alkenyl, aryl, heteroaryl, cycloalkyl, heterocyclo, or C(═O)R₂₈; or when G is NH(C═O)R₁₉, R₁₉ may be a bond joined to W to define a heterocyclo ring;

R₂₁ and R₂₂ are selected from hydrogen, alkyl, and substituted alkyl;

R₂₃ and R₂₄ are independently selected from hydrogen, alkyl, substituted alkyl, aryl, heteroaryl, heterocyclo, and cycloalkyl;

R₂₅, R₂₆ and R₂₇ are independently selected from hydrogen, alkyl, substituted alkyl, cycloalkyl, aryl, heterocyclo, and heteroaryl; or R₂₅ and R₂₆ may join together to form a heterocyclo or heteroaryl, except R₂₆ is not hydrogen when joined to a sulfonyl group as in —S(O)ₚR₂₆ or —NR₂₅SO₂R₂₆;

R₂₈ is hydrogen, alkyl, or substituted alkyl;

n is 0, 1, 2, 3 or 4;

p is 1, 2, or 3;

r and s are 0 or 1;

x is 0, 1, or 2;

y is 0,1, 2, 3 or 4; and

z is 0, 1, or 2.

More preferred melanocortin receptor agonists that may be used in the inventive combination is a compound having the formula,

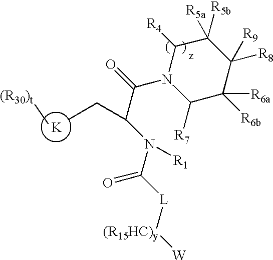

or a pharmaceutically-acceptable salt, hydrate, or prodrug thereof, in which:

R₁ is hydrogen or C₁₋₄alkyl;

R₁₅ is hydrogen, C₁₋₄alkyl, or substituted C₁₋₄alkyl,

K is aryl or heteroaryl;

R₃₀ is C₁₋₄alkyl, hydroxy, methoxy, ethoxy, halogen, nitro, cyano, amino, C₁₋₄alkylamino, phenyl, or C(═O)phenyl; tis0, 1, or 2;

z is 0 or 1, and L, W, and R₄-R₉ are as defined above.

Preferred methods and compositions of this invention comprise use of at least one compound that is a selective MC-1R agonist or MC-4R agonist as described in U.S. patent applications Serial Nos. 60/273,206, and 60/273,291, filed Mar. 2, 2001, the entire contents of which are incorporated herein by reference, as well as corresponding non-provisional patent applications Ser. nos._______, filed Mar. 4, 2002, also incorporated herein.

Further preferred melanocortin-receptor agonists to be used in the inventive combination are described hereinafter. However, it is to be understood that these examples are non-limiting, and the inventive co-administration pertains more generally to use of any compound that is an MC-1R agonist or an MC-4R agonist together with a CAMP-PDE inhibitor, particularly a selective MC-1R agonist or selective MC-4R agonist.

### Cyclic AMP PDE Inhibitors

The cAMP-PDE inhibitor used according to the invention may comprise at least one PDE1 inhibitor (including those described in Journal of Medicinal Chemistry, Vol. 40, pp. 2196-2210 [1995]), PDE2 inhibitor (including hydroxynonyladenine), PDE3 inhibitor (including revizinone, pimobendan, olprinone, milrinone, and motapizone), PDE4 inhibitor (including ariflo, rolipram, cilomilast, piclamilast, and Ro-20-1724), and/or PDE7 inhibitor. IBMX, a dual inhibitor of cAMP and cGMP PDEs, and inhibitors of PDE 8 (such as dipyridamole) and/or PDEs 10 and 11, are also contemplated as within the scope of the invention. However, use of PDE 3, 4, 7 and 8 inhibitors is preferred.

The methods and compositions of this invention may comprise use of one or more cAMP-PDE inhibitors described in one or more of the following U.S. patents, each of which is incorporated herein by reference: U.S. Pat. Nos. 6,211,222 and 6,127,398, “Substituted indazole derivatives and related compounds; U.S. Pat. No. 6,211,203, “Benzofuran-4-carboxamides”; U.S. Pat. No. 6,200,993, “Heterosubstituted pyridine derivatives as PDE4 inhibitors”; U.S. Pat. No. 6,191,138, “Phenanthridines”; U.S. Pat. No. 6,180,650, “Heterosubstituted pyridine derivatives as PDE4 inhibitors”; U.S. Pat. No. 6,136,821, “Naphthyridine derivatives”; U.S. Pat. No. 6,054,475, “Substituted dihydrobenzofuran-based phosphodiesterase 4 Inhibitors useful for treating airway disorders”; U.S. Pat. No. 6,043,263, “(2,3-dihydrobenzofuranyl)-thiazoles as phosphodiesterase inhibitors”; U.S. Pat. No. 6,011,037, “Thiazole derivatives with phosphodiesterase-inhibiting action”; U.S. Pat. No. 5,972,927, “Diazepinoindoles as phosphodiesterase 4 inhibitors”; U.S. Pat. No. 5,919,801, “N-substituted piperidines as PDE4 inhibitors”; U.S. Pat. No. 6,204,275, “PDE IV Inhibiting compounds, compositions and methods of treatment”; U.S. Pat. No. 6,143,782, “Anti-inflammatory and anti-asthma treatment with reduced side effects”; U.S. Pat. No. 6,103,749, “Aryl imidazole compounds having phosphodiesterase IV activity”; U.S. Pat. No. 6,096,768, “Compounds containing phenyl linked to aryl or heteroaryl by an aliphatic or heteroatom containing linking group”; U.S. Pat. No. 6,075,016, “6,5-fused aromatic ring systems having enhanced phosphodiesterase IV inhibitory activity”; U.S. Pat. No. 6,040,447, “Purine compounds having PDE IV inhibitory activity and methods of synthesis”; U.S. Pat. No. 6,034,089, “Aryl thiophene derivatives as PDE IV inhibitors”; U.S. Pat. No. 6,020,339, “Aryl furan derivatives as PDE IV inhibitors”; U.S. Pat. No. 5,935,978, “Compounds containing phenyl linked to aryl or heteroaryl by an aliphatic or heteroatom containing linking group”; U.S. Pat. No. 5,935,977, “Substituted vinyl pyridine derivative and drugs contaning same”; U.S. Pat. No. 5,840,724, “Compounds containing phenyl linked to aryl or heteroaryl by an aliphatic or heteroatom containing linking group”; U.S. Pat. No. 5,710,170, “Tri-aryl ethane derivatives as PDE IV inhibitors”; U.S. Pat. No. 5,710,160, “Diphenyl pyridyl ethane derivatives as PDE IV inhibitors”; U.S. Pat. No. 5,698,711, “Compounds containing phenyl linked to aryl or heteroaryl by an aliphatic or heteroatom containing linking group”; U.S. Pat. No. 5,691,376, “Substituted biphenyl derivatives”; U.S. Pat. No. 5,679,696, “Compounds containing phenyl linked to aryl or heteroaryl by an aliphatic or heteroatom containing linking group”; U.S. Pat. No. 5,665,737, “Substituted benzoxazoles”; U.S. Pat. No. 5,650,444, “Substituted biphenyl derivatives”; U.S. Pat. No. 5,616,614, “Naphthylalkylamines”; U.S. Pat. No. 5,541,219, “1-Alkoxy-2-(alkoxy or cycloalkoxy)-4-(cyclothioalkyl or cyclothioalkenyl)benzenes as inhibitors of cyclic AMP phosphodiesterase and tumor necrosis factor”; U.S. Pat. No. 5,502,072, “Substituted oxindoles”; U.S. Pat. No. 5,466,697, “8-phenyl-1,6-naphthyridine-5-ones”; U.S. Pat. No. 5,459,151, “N-acyl substituted phenyl piperidines as bronchodilators and antiinflammatory agents”; U.S. Pat. No. 5,393,788, “Phenylalkyl oxamides”; U.S. Pat. No. 5,356,923, “1-hydroxy-4(3-cyclopentyloxy-4-methoxyphenyl)-2-pyrrolidone and anti-hypertensive use thereof”; U.S. Pat. No. 5,250,700, “Phenyl pyrazolidinones as bronchodilators and anti-inflammatory agents”; U.S. Pat. No.5,191,084, “Phenyl pyrazolidinones as bronchodilators and anti-inflammatory agents”; U.S. Pat. No. 5,124,455, “Oxime carbamates and oxime carbonates as bronchodilators and anti inflammatory agents”; U.S. Pat. No. 6,180,791, “Synthesis of 8-substituted xanthines”; U.S. Pat. No. 6,057,369, “Substituted (aryl, heteroaryl, arylmethyl or heteroarylmethyl)hydroxamic acid compounds”; U.S. Pat. No. 5,541,219, “1-Alkoxy-2-(alkoxy or cycloalkoxy)-4-(cyclothioalkyl or cyclothioalkenyl)benzenes as inhibitors of cyclic AMP phosphodiesterase and tumor necrosis factor”; U.S. Pat. No. 5,362,915, “Phenyl substituted cycloalkenyl compounds useful as PDE IV inhibitors”; U.S. Pat. No. 6,040,329, “Substituted indazole analogs”; U.S. Pat. No.5,958,953, “Substituted indazole derivatives”; U.S. Pat. No. 6,090,817, “Phenylpyridine derivatives useful as phosphodiesterase inhibitors”; U.S. Pat. No. 5,922,740, “Heterocyclylcarbonyl substituted benzofuranylureas”; U.S. Pat. No. 5,866,571, “9-substituted 2-2-n-alkoxyphenyl)-purin-6-ones”; U.S. Pat. No. 5,861,404, “2,9-disubstituted purin-6-ones”; U.S. Pat. No. 5,861,396, “Purin-6-one derivatives”; U.S. Pat. No. 5,721,238, “2,8-disubstituted quinazolinones”; U.S. Pat. No. 5,723,463, “Pyrido 3,2-Pyrazinones with Anti-asthmatic action and Processes for their Manufacture”; and U.S. Pat. No. 5,596,013, “Dihydro pyrazolopyrroles.”

Preferred cAMP-PDE Inhibitors comprise PDE4 inhibitors, more particularly compounds that demonstrate greater inhibition of LPDE4 than HPDE4, and also inhibit PDE4 preferentially to other known types of PDEs, such as PDE1, PDE2, and PDE3.

PDE type 7 inhibitors that may be used according to the invention include compounds described in WO 01/029049, “Imidazole derivatives as Phophodiesterase VII Inhibitors,” by Merck; WO 00/068230, “9-(1,2,3,4-tetrahydronapthalen-1-yl)-1,9-dihydropurin-6-one Derivatives as PDE7 Inhibitors” by Darwin Discovery Ltd; WO 00/014083 to Inflazyme Pharmaceuticals, Ltd; Martinez et al., “Benzyl Derivatives of 2,1,3-Benzo and Benzothieno (3,2-a)thiadiazine 2,2 dioxides: first Phosphodiesterase 7 Inhibitors,” J. Med. Chem. Vol. 43 (2000), at pp 683-89; Barnes et al., “Synthesis and Structure-Activity Relationships of Guanine Analogues as Phosphodiesterase 7 (PDE 7) Inhibitors,” Biorg. Med. Chem. Lett. Vol. 11(8) (2001) at pp. 1081-83; and the compound designated AWD 12187 by ASTA Medica (Germany). Each of the patents and publications referred to above is incorporated herein by reference.

### Methods of Preparation

### Melanocortin-receptor Agonists

Melanocortin-receptor agonists for use in the inventive methods and compositions may be prepared by methods illustrated in the following Schemes I to III. Starting materials are commercially available or can be readily prepared by one of ordinary skill in the art using known methods. Solvents, temperatures, pressures, and other reaction conditions may readily be selected by one of ordinary skill in the art. High Speed Analoging (HSA) may be employed in the preparation of compounds, for example, where the intermediates possess a carboxylic acid or amino group.

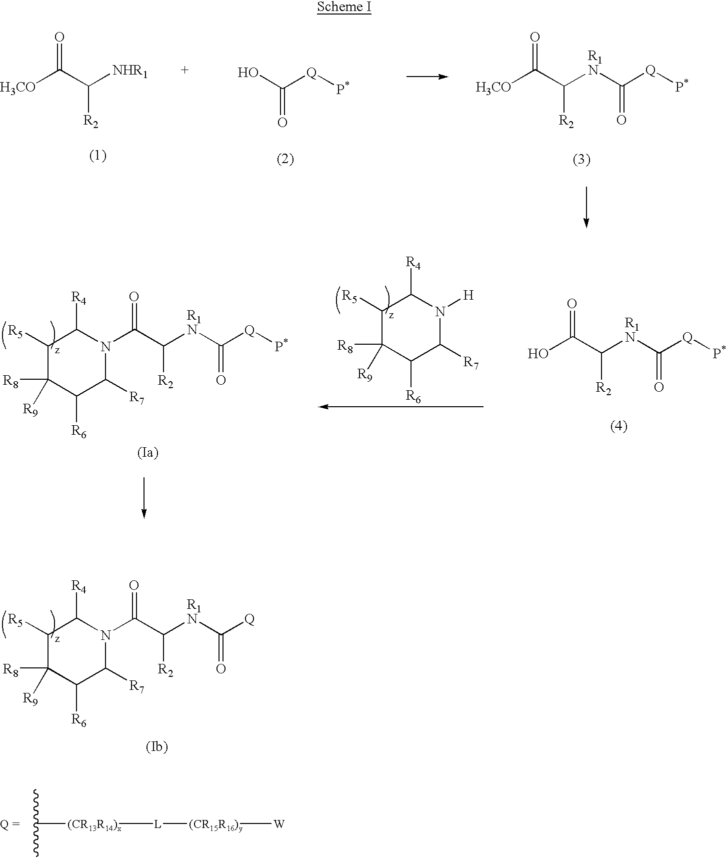

Compounds of formula (Ib) can be prepared from compounds (Ia) [wherein P* is an amino protecting group, such as -Boc-, -CBZ-, -Fmoc-, which can be present in Q as in formula (Ia) or independently bonded to Q] via an appropriate amine deprotection process in an inert solvent at a temperature in the range −10° C. to 100° C. The choice of deprotection routes can be chosen by one of ordinary skill in the art. They include, but are not limited to TFA or hydrogen chloride acid for -Boc-, hydrogenation with an appropriate metal catalyst (such as Pd), for -CBZ-, or a base, such as NMM or DEA, for -Fmoc-. Inert solvents include, but are not limited to methylene dichloride, alcoholic solvents, THF, acetic acid, DMF, acetonitrile, and dioxane.

Compounds of formula (Ia) can be prepared by the coupling of compounds of formula (5) with compounds (4) using an appropriate carboxylic acid activating reagent in an inert solvent. Exemplary carboxylic acid activating agents include carbonyldiimidazole, dicyclohexylcarbodiimide, pentofluorophenol trifluoroacetate, 1-(3-dimethylaminopropyl)-3-ethylcarbodiimide, or other activating agents known by one of ordinary skill in the art. Exemplary inert solvents include ethers, including THF and dioxane, DMF, acetonitrile, or CH₂Cl₂.

Compounds (4) can be prepared by the hydrolysis of compounds (3) using a hydroxide source. Exemplary hydroxide sources include NaOH or LiOH. Exemplary solvents include water, alcohols, and mixtures of ethers/water.

Compounds (3) can be prepared by the coupling of compounds (1) and (2) using an appropriate carboxylic acid activating reagent in an inert solvent. Exemplary carboxylic acid activating agents include carbonyidiimidazole, dicyclohexylcarbodiimide, pentofluorophenol trifluoroacetate, 1-(3-dimethylaminopropyl)-3-ethylcarbodiimide, or other activating agents known by one of ordinary skill in the art. Exemplary inert solvents include ethers, including THF and dioxane, DMF, acetonitrile, or CH₂Cl₂.

Compounds (1), (2) and (3) are either commercially available or available by methods known to one of ordinary skill in the art.

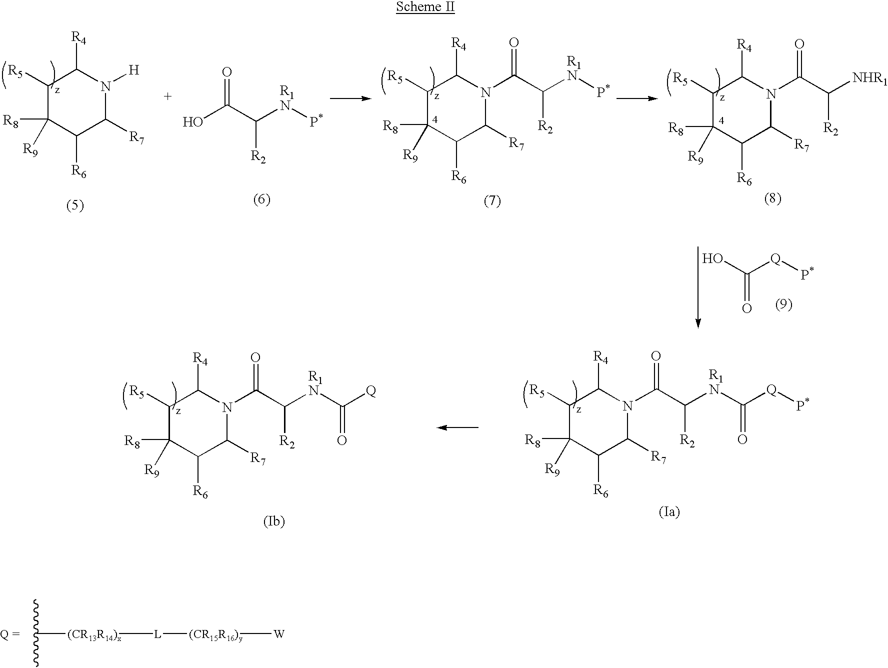

Compounds of formula (Ib) can be prepared from compounds of formula (Ia) [wherein P* is an amino-protecting group as in Scheme I] via an appropriate amine deprotection process in an inert solvent at a temperature in the range from −10° C. to 100° C. The choice of deprotection routes can be chosen by one of ordinary skill in the art. They include, but are not limited to TFA or hydrogen chloride acid for -Boc-, hydrogenation with an appropriate metal catalyst for -CBZ-, or a base, such as NMM or DEA, for -Fmoc-. Inert solvents include, but are not limited to methylene dichloride, alcoholic solvents, THF, acetic acid, DMF, acetonitrile, and dioxane.

Compounds of formula (Ia) can be prepared by the coupling of compounds (8) and (9) using an appropriate carboxylic acid activating reagent in an inert solvent. Exemplary carboxylic acid activating agents include carbonyldiimidazole, dicyclohexylcarbodiimide, pentofluorophenol trifluoroacetate, 1-(3-dimethylaminopropyl)-3-ethylcarbodiimide, or other activating agents known by on of ordinary skill in the art. Exemplary inert solvents include ethers, including THF and dioxane, DMF, acetonitrile, or CH₂Cl₂.

Compounds (8) [wherein P* is an amino-protecting group as above] can be prepared from compounds (7) via an appropriate amine deprotection process in an inert solvent at temperatures ranging from −10° C. to 100° C. The choice of deprotection routes can be chosen by one of ordinary skill in the art and include those referenced above in Scheme I for -Boc-, -CBZ-, and -Fmoc-. Inert solvents include, but are not limited to methylene dichloride, alcoholic solvents, THF, acetic acid, DMF, acetonitrile, and dioxane.

Compounds (7) can be prepared by the coupling of compounds (5) and (6) using an appropriate carboxylic acid activating reagent in an inert solvent. Exemplary carboxylic acid activating agents include carbonyldiimidazole, dicyclohexylcarbodiimide, pentofluorophenol trifluoroacetate, 1-(3-dimethylaminopropyl)-3-ethylcarbodiimide, or other activating agents known by one of ordinary skill in the art. Exemplary inert solvents include ethers, including THF and dioxane, DMF, acetonitrile, or CH₂Cl₂.

Compounds (5) and (6) are either commercially available or available by methods known to one of ordinary skill in the art.

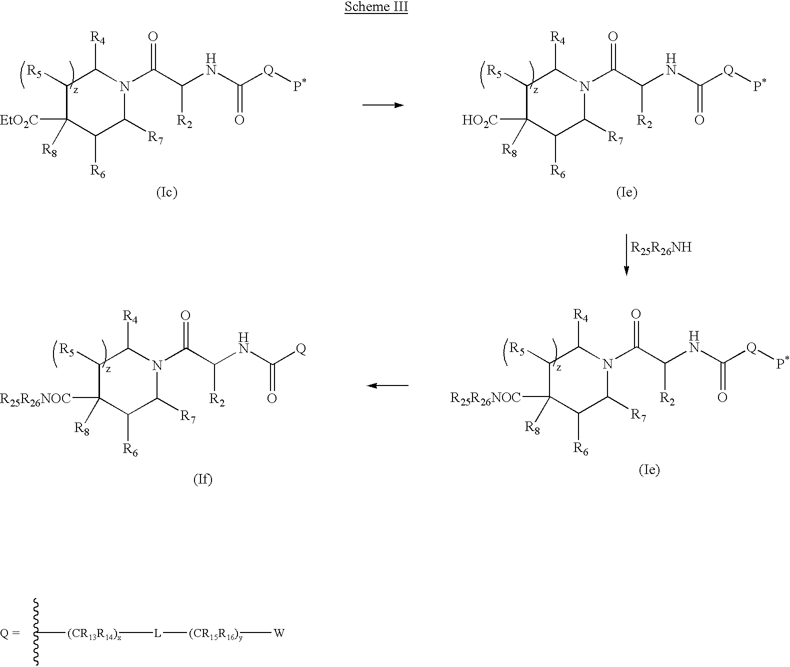

Compounds of formula (If) can be prepared from compounds of formula (Ie) [wherein P* is an amino protecting group as in Scheme I] via an appropriate amine deprotection process chosen by one of ordinary skill in the art, such as described above in Schemes I and II.

Compounds of formula (Ie) can be prepared by the coupling of compounds of formula (Id) with amines of the formula R₂₅R₂₆NH using an appropriate carboxylic acid activating reagent in an inert solvent. Exemplary carboxylic acid activating agents include carbonyidiimidazole, dicyclohexylcarbodiimide, pentofluorophenol trifluoroacetate, 1-(3-dimethylaminopropyl)-3-ethylcarbodiimide, or other activating agents known by one of ordinary skill in the art. Exemplary inert solvents include ethers, including THF and dioxane, DMF, acetonitrile, or CH₂Cl₂.

Compounds of formula (Id) can be prepared by the hydrolysis of compounds of formula (Ic) using a hydroxide source. Exemplary hydroxide sources include NaOH or LiOH. Exemplary solvents include water, alcohols, and mixtures of ethers/water.

Amines of the formula R₂₅R₂₆NH are either commercially available or available by methods known to one of ordinary skill in the art. Compounds of formula (Ic) can be prepared as described above in Schemes I and II.

All documents cited in the present specification are incorporated herein by reference in their entirety.

### Cyclic AMP-PDE Inhibitors

Methods for preparing cAMP-PDE inhibitors to be used according to the invention are described in the U.S. and International patents and articles cited above, which are incorporated herein by refernece. Further methods are described in U.S. Pat. Nos. 5,856,498, 5,808,082, 5,728,838, each of which is titled “Method of preparing phosphodiesterase IV inhibitors”, and in Drugs of the Future, “SB-207499, Ariflo” Vol. 23, No. 6 (1998), pp. 607-615, incorporated herein by reference.

### Utility

The methods and compositions of this invention may be used as anti-inflammatory, anti-asthmatic, anti-thrombotic, anti-despressive, and/or neurogenerative treatments and agents. The co-administration of at least one melanocortin-receptor agonist and at least one cAMP-PDE inhibitor according to the invention is particularly useful in treating inflammation characterized by the activation of NF-κB and/or release of inflammatory cytokines. The inventive co-administration can have multiple effects on cells of the immune system, including altering the expression of immune related genes including cytokines, adhesion molecules, and nitric oxide synthase. The co-administration of at least one MC-1R agonist, MC-4R agonist, and at least one cAMP-PDE inhibitor according to the invention is particularly useful in treating stroke, stroke and other ischemic brain diseases and/or neurodegeneration associated therewith, and the neurodegeneration of, and consequences of, traumatic brain injury. As used herein, the term “treating” or “treatment” refers to prophylaxis measures designed to inhibit or delay the onset of the disease or disorder and to responsive measures designed to alleviate, ameliorate, lessen, or cure the disease or disorder and/or its symptoms.

The inventive co-administration is designed to elevate cAMP levels in cells and maintain the enhanced cAMP levels which is believed to effect an inhibition of NF-κB activity. In view of this activity, the invention will be useful in treating consequences of many diseases associated with chronic and acute inflammation and immune-modulation. Such diseases include, but are not limited to, inflammatory bowel disease, irritable bowel syndrome, gall bladder disease, Chrohn's disease, rheumatoid arthritis, osteoarthritis, osteoporosis, traumatic arthritis, rubella arthritis, muscle degeneration, pancreatis (acute or chronic), psoriasis, glomerulonephritis, serum sickness, lupus (systematic lupus erythematosis), urticaria, scleraclerma, schleroderma, chronic thyroiditis, Grave's disease, dermatitis (contact or atopic), dermatomyositis, alopecia, atopic eczemas, ichthyosis, fever, sepsis, migraine, cluster headaches, Alzheimer's Disease, Parkinson's disease, Creutzfeldt-Jacob disease, multiple sclerosis, tuberculosis, dementia, and transplant or graft-host rejections (e.g., kidney, liver, heart, lung, pancreas, bone marrow, cornea, small bowel, skin allografts, skin homografts and heterografts, etc.). The compounds may also be used to treat respiratory allergies and diseases including asthma, acute respiratory distress syndrome, hayfever, allergic rhinitis, and chronic obstructive pulmonary disease; and inflammatory disorders of the central nervous system, including HIV encephalitis, cerebral malaria, meningitis, and ataxia telangiectasis. Additionally, the compounds may be useful in treating pain, e.g., post-operative pain, neuromuscular pain, headache, pain caused by cancer, dental pain, and arthritis pain.

In view of their activity in inhibiting NF-κB activity, the compounds may be used to treat viral and autoimmune diseases including herpes simplex type 1 (HSV-1), herpes simplex type 2 (HSV-2), cytomegalovirus, Epstein-Barr, human immunodeficiency virus (HIV), Addison's disease (autoimmune disease of the adrenal glands), idiopathic adrenal insufficiency, autoimmune polyglandular disease (also known as autoimmune polyglandular syndrome), chronic active hepatitis or acute hepatitis infection (including hepatitis A, hepatits B, and hepatitis C), autoimmune gastritis, autoimmune hemolytic anemia, and autoimmune neutropenia. The compounds of the invention may also be used to treat fungal infections such as mycosis fungoides.

In addition, the compounds of this invention are useful in treating diseases of the cardiovascular system including those diseases in which inflammation is an underlying component. These diseases include but are not limited to atherosclerosis, transplant atherosclerosis, peripheral vascular disease, inflammatory vascular disease, intermittent claudication, restenosis, cerebrovascular stroke, transient ischemic attack, myocardial ischemia and myocardial infarction. The compounds also may be used to treat hypertension, hyperlipidemia, coronary artery disease, unstable angina, thrombosis, thrombin-induced platelet aggregation, and/or consequences occurring from thrombosis and/or the formation of atherosclerotic plaques.

Additionally, the compounds may be useful to treat stroke and other ischemic brain diseases and/or neurodegeneration associated therewith, and the neurodegeneration of, and consequences of, traumatic brain injury.

In view of their ability to act as immunomodulators in the skin and affect the production of melanin in the skin, these compounds are useful in altering pigmentation in the skin and may be used as photoprotective agents including agents for preventing, treating, or ameliorating sunburn. The compounds also may be used in treating acne, vitiligo, alopecia arreata, photosensitivity disorders, albinism, and porphyria. Addditionally, the compounds are useful to promote cosmetic as well as therapeutic tanning.

The compounds of the invention may also be used to treat neurodegenerative disorders including depression, anxiety, compulsion (obsessive-compulsive disorder), neuroses, psychosis, insomnia/sleep disorder, sleep apnea, and drug or substance abuse.

The compounds of the invention may be used to treat male or female sexual dysfunction. Male sexual dysfunction includes impotence, loss of libido, and erectile dysfunction (including but not limited to ejaculatory failure, premature ejaculation, or an inability to achieve or maintain an erection or to achieve an orgasm). Female sexual dysfunction may include sexual arousal disorder or disorders relating to desire, sexual receptivity, orgasm, and/or disturbances in trigger points of sexual function. Female sexual dysfunction may also include sexual pain, premature labor, dysmenorrhea, excessive menstruation, and endometriosis.

The compounds of the invention may also be used to treat bodyweight disorders including but not limited to obesity and anorexia (e.g., by altering appetite, metabolic rate, fat intake or carbohydrate craving); and diabetes mellitus (by enhancing glucose tolerance and/or decreasing insulin resistance).

The compounds also may be used to treat cancer, more particularly, cancer of the lung, prostate, colon, breast, ovaries, and bone, or angiogenic disorders including the formation or growth of solid tumors.

The compounds of the invention may also be used to treat veterinary disease such as veterinary viral infections, including feline immunodeficiency virus, bovine immunodeficiency virus, and canine immunodeficiency virus.

The term “melanocortin-receptor associated condition” and the term “cAMP-associated condition” when used herein refers to each of the above-referenced conditions, disorders, or diseases that may be treated by activating MC-1R and/or MC-4R, inhibiting cAMP-PDE, and/or modulating intracellular levels of cAMP, as if each of these conditions, disorders and diseases was set forth herein at length.

Other therapeutic agents may be used along with the at least one MC-1R agonist, MC-4R agonist, and cAMP-PDE inhibitor according to the invention. Such other therapeutic agents include anti-inflammatory agents antibiotics, anti-viral agents, anti-fungal agents, anti-diabetic agents, anti-osteoporosis agents, anti-obesity agents or appetite suppressants, growth promoting agents (including growth hormone secretagogues), anti-anxiety agents, anti-depressants, anti-hypertensive agents, cholesterol/lipid lowering agents, bone resorption inhibitors, and anti-tumor agents including antiproliferative agents, or cytotoxic drugs.

Examples of suitable other anti-inflammatory agents with which the inventive compounds may be used include aspirin, non-steroidal antiinflammatory drugs (NSAIDs) (such as ibuprofen and naproxin), TNF-α inhibitors (such as tenidap and rapamycin or derivatives thereof), or TNF-α antagonists (e.g., infliximab, OR1384), prednisone, dexamethasone, Enbre®, cyclooxygenase inhibitors (i.e., COX-1 and/or COX-2 inhibitors such as Naproxen®, Celebrex®, or Vioxx®), CTLA4-lg agonists/antagonists, CD40 ligand antagonists, IMPDH inhibitors, such as mycophenolate (CellCept®), integrin antagonists, alpha-4 beta-7 integrin antagonists, cell adhesion inhibitors, interferon gamma antagonists, ICAM-1, prostaglandin synthesis inhibitors, budesonide, clofazimine, CNI-1493,° C.D4 antagonists (e.g., priliximab), p38 mitogen-activated protein kinase inhibitors, protein tyrosine kinase (PTK) inhibitors, IKK inhibitors, therapies for the treatment of irritable bowel syndrome (e.g., Zelmac® and Maxi-K® openers such as those disclosed in U.S. Pat. No. 6,184,231 B1), or other NF-κB inhibitors, such as corticosteroids, calphostin, CSAIDs, 4-substituted imidazo [1,2-A]quinoxalines as disclosed in U.S. Pat. No. 4,200,750; Interleukin-10, glucocorticoids, salicylates, nitric oxide, and other immunosuppressants; and nuclear translocation inhibitors, such as deoxyspergualin (DSG). To treat pain such as migraine and other headaches, the inventive compounds may be used in combination with aspirin, NSAIDs, or with 5-HT_{ID} receptor agonists such as sumitriptan, eletriptan or rizatriptan.

Examples of suitable other antibiotics with which the inventive compounds may be used include β-lactams (e.g., penicillins, cephalosporins and carbopenams); β-lactam and lactamase inhibitors (e.g., augamentin); aminoglycosides (e.g., tobramycin and streptomycin); macrolides (e.g., erythromycin and azithromycin); quinolones (e.g., cipro and tequin); peptides and deptopeptides (e.g. vancomycin, synercid and daptomycin) metabolite-based anti-biotics (e.g., sulfonamides and trimethoprim); polyring systems (e.g., tetracyclins and rifampins); protein synthesis inhibitors (e.g., zyvox, chlorophenicol, clindamycin, etc.); and nitro-class antibiotics (e.g., nitrofurans and nitroimidazoles).

Examples of suitable other antifungal agents with which the inventive compounds may be used include fungal cell wall inhibitors (e.g., candidas), azoles (e.g., fluoconazole and vericonazole), and membrane disruptors (e.g., amphotericin B).

Examples of suitable other antiviral agents for use with the inventive compounds include nucleoside-based inhibitors, protease-based inhibitors, and viral-assembly inhibitors.

Examples of suitable anti-diabetic agents for use in combination with the compounds of the present invention include biguanides (e.g., metformin or phenformin), glucosidase inhibitors (e.g,. acarbose or miglitol), insulins (including insulin secretagogues, sensitizers or mimetics), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, SGLT2 inhibitors, glycogen phosphorylase inhibitors, inhibitors of fatty acid binding protein (aP2), glucagon-like peptide-1 (GLP-1), dipeptidyl peptidase IV (DP4) inhibitors, Alistat®, Meridia®, and Zenacol®.

Examples of suitable anti-osteoporosis agents for use in combination with the compounds of the present invention include alendronate, risedronate, PTH, PTH fragment, raloxifene, calcitonin, RANK ligand antagonists, calcium sensing receptor antagonists, TRAP inhibitors, selective estrogen receptor modulators (SERM) and AP-1 inhibitors.

Examples of suitable anti-obesity agents for use in combination with the compounds of the present invention include aP2 inhibitors, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin, adrenergic (and dopamine) reuptake inhibitor, such as sibutramine, topiramate (Johnson & Johnson) or axokine (Regeneron), other thyroid receptor beta drugs, such as a thyroid receptor ligand as disclosed in WO 97/21993 (U. Cal SF), WO 99/00353 (KaroBio) and GB98/284425 (KaroBio), and/or an anorectic agent (such as dexamphetamine, phentermine, phenylpropanolamine or mazindol). Additionally, the inventive compounds may be used with an α-gluocosidase inhibitor, an MHG-CoA reductase inhibitor, a sequestrant chlolestoral lowering agent, a β3 adrenergic receptor agonist, a neuropeptide Y antagonist, or an α2-adrenergic receptor antagonist.

A still further use of the compounds of the invention is in combination with estrogen, testosterone, a selective estrogen receptor modulator, such as tamoxifen or raloxifene, or other androgen receptor modulators.

A further use of the compounds of this invention is in combination with steriodal or non-steroidal progesterone receptor agonists (“PRA”), such as levonorgestrel, medroxyprogesterone acetate (MPA).

Examples of suitable anti-anxiety agents for use in combination with the compounds of the present invention include benzodiazepines, diazepam, lorazepam, buspirone (Serzone®), oxazepam, and hydroxyzine pamoate, or dopamine recetpor agonists.

Examples of suitable anti-depressants for use in combination with the compounds of the present invention include citalopram, fluoxetine, nefazodone, sertraline, and paroxetine.

In treating skin disorders or diseases as described above, the compounds may be used alone or in combination with a retinoid, such as tretinoin, or a vitamin D analog.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, and spironolactone), renin inhibitors, ACE inhibitors (e.g., captopril, Vanlev®, pravachol, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), nitrates, and cardiac glycosides (e.g., digitalis and ouabain).

Examples of suitable cholesterol/lipid lowering agents for use in combination with the compounds of the present invention include HMG-CoA reductase inhibitors, squalene synthetase inhibitors, fibrates, bile acid sequestrants, ACAT inhibitors, MTP inhibitors, lipooxygenase inhibitors, an ileal Na⁺/bile acid cotransporter inhibitor, cholesterol absorption inhibitors, and cholesterol ester transfer protein inhibitors (e.g., CP-529414).

The above other therapeutic agents, when employed in combination with the co-administration of the present invention, may be used, for example, in those amounts indicated in the Physicians′ Desk Reference (PDR) or as otherwise determined by one of ordinary skill in the art.

The melanocortin-receptor agonist (MC-1R or MC-4R) and cAMP-PDE inhibitor may be formulated together, in a single carrier or single dosage unit (e.g., combined in one compartmentalized or non-compartmentalized capsule or tablet, or combined in one powder, liquid, gel, and so forth). When the melanocortin-receptor agonist and cAMP-PDE inhibitor are not formulated together, either agent may be administered first, or they may be administered alternatively, or they may be formulated separately and administered simultaneously. Since an advantage of the cAMP-PDE inhibitor involves counteracting an overactive PDE response upon administering a melanocortin-receptor agonist, this advantage can be achieved when administration of the melanocortin-receptor agonist is delayed after the cAMP-PDE inhibitor is administered. When not administered at the same time, it is preferred that at least one cAMP-PDE inhibitor be administered followed by at least one melanocortin-receptor agonist administered within about four hours thereafter.

The following description of pharmaceutical compositions is intended to refer to formulations for either or both of the melanocortin-receptor agonist and cAMP-PDE inhibitor.

Pharmaceutical compositions may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The melanocortin-receptor agonist and/or cAMP-PDE inhibitor may be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compositions may be delivered orally, such as in the form of tablets, capsules, granules, powders, or liquid formulations including syrups; topically, such as in the form of solutions, suspensions, gels or ointments; sublingually; bucally; parenterally, such as by subcutaneous, intravenous, intramuscular or intrastemal injection or infusion techniques (e.g., as sterile injectable aqueous or non-aqueous solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compositions may be administered in a form suitable for immediate release or extended release. Immediate release or extended release may be achieved with suitable pharmaceutical compositions or, particularly in the case of extended release, with devices such as subcutaneous implants or osmotic pumps. It is possible that only one of the agents, e.g., melanocortin-receptor agonist or cAMP-PDE inhibitor, will be delivered via a sustained release mechanism. For example, one agent may be included in a tablet and coated with a sustained release material, with the other agent included in the same tablet but having a different or no coating, to control the release of the combined agents in the gastrointestinal tract and/or control interaction between the two agents before they are absorbed by the patient.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compositions may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use. The compositions may be used in combination with one or more surfactants, such as a recominant surfactant protein C based surfactant (rSP-C).

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The combination of melanocortin-receptor agonist and cAMP-PDE inhibitor may be formulated such that, although the active ingredients are combined in a single dosage unit, the contact between the two ingredients is minimized. This may be accomplished as described above with coatings designed to regulate the timing at which the ingredients are released into a patient's gastrointestinal tract. Another approach would be to provide enteric or polymeric coatings or layers between the components. Also, different modes of administration may be used for the two or more components. For example, one component could be intravenously administered, while the other is administered via tablet or capsule. Any combination of the foregoing modes of administration may be used.

The amount of melanocortin-receptor agonist and cAMP-PDE inhibitor to be included in the inventive combination, including the relative amounts of each component, may be varied and will depend upon a variety of factors, including the activity of the specific compounds employed, the metabolic stability and length of action of the compounds, the species, age, body weight, general health, sex and diet of the subject, the mode and timing of administration(s), rate of excretion, combination with other drugs, and severity of the particular condition. The desired therapeutically-effective amount of the compounds in combination may be determined by one of ordinary skill in the art, and includes exemplary dosage amounts for a mammal of from about 0.01 to 100 mg/kg of body weight of each active compound per day, which may be administered in a single dose or in the form of individual divided doses, such as from 1 to 4 times per day. Preferred subjects for treatment include animals, most preferably mammalian species such as humans, and domestic animals such as dogs, cats, horses, and the like, subject to cAMP-associated conditions.

The compounds particularly described herein for use in the inventive combination have been tested and have measurable activity as agonists of MC-1R and/or MC-1R according to an assay described below and/or an assay known in the field, such as, for example, assays described in WO 00/74679 A1 and WO 01/91752.

### Assays

### MC-1R

HBL cells, a human melanoma cell line licensed from Prof. G. Ghanem (Lab. of Oncology & Exp. Surgery, Free University of Brussels, Brussels, Belgium) were used as a source of the human MC-1R. cAMP was measured using the cAMP SPA Direct Screening Assay System from Amersham (RPA 559). 20,000 HBL cells were plated into each well of a half-area 96 well white plate and were used between 16-48 hours after plating. Cells were incubated at 37° C. for 15 minutes in 25 μM IBMX to inhibit phosphodieterase activity. As per kit instructions, Assay Buffer Concentrate was diluted 1 to 50 with dH₂O to prepare Assay Buffer (50 mM acetate buffer containing 0.01% sodium azide). Vials containing rabbit anti-succinyl cAMP serum and the tracer, adenosine 3′,5′-cyclic phosphoric acid 2′-0-succinyl-3-[¹²⁵I] iodotyrosine methyl ester, were resuspended with 7.5 ml Assay Buffer. SPA anti-rabbit reagent (donkey anti-rabbit IgG coupled to SPA PVT beads) was resuspended with 15 ml Assay Buffer. All reagents were stored at 4° C. after reconstitution. Melanocortin ligands or compounds were prepared in DMSO and added to the IBMX-treated cells as 100×concentrated stocks. 50 nM α-MSH was used for the maximum response and 1 ul DMSO was included in the negative control wells. The final concentration of DMSO was 1% in all the samples. After 15-30 minutes of stimulation, the reaction was terminated by the aspiration of the contents of the well followed by addition of 15 ul Assay Buffer containing 0.1 N HCl. Plates were kept at room temperature for at least 30 minutes to effect extraction of cAMP. Antiserum, Tracer, and SPA anti-rabbit reagent solutions were mixed 1:1:1 just prior to use. 15 ul of SPA reagent mixture was dispensed into each well and plates were incubated at room temperature for a minimum of 5 hours. Plates were subsequently counted for 6 minutes per sample in a TopCount scintillation reader with background subtraction. Data was analyzed in relation to a cAMP standard curve.

### MC-4R

A. Binding Assay

The membrane binding assay may be used to identify competitive inhibitors of [¹²⁵I]NDP-α-MSH binding to cloned human MC4R expressed in Hi5 insect cells infected by a baculovirus/human MC4R receptor construct.

Hi5 cells are grown in suspension in Express Five SFM Insect Cell Media (Gibco, Cat. No. 10486-025) at 27° C. with constant shaking. Hi5 cells are infected using the following protocol:

Cells at a density of 1×10⁶ cells/mL are spun down at 1000 rpm (Beckman GS-6KR centrifuge) for 10 minutes.

Cells are resuspended in 10% of their original volume in a sterile 50 mL conical centrifuge tube wrapped with aluminum foil. Virus is added at a Multiplicity of Infection (MOI) of 3 and incubated for 1 hour at room temperature with gentle shaking.

This cell/virus mix is added to the appropriate volume of medium to attain the original volume and incubated at 27° C. with constant shaking for 72 hours.

Cells are spun down in 50 mL conical centrifuge tubes at 1000 rpm for 10 minutes. Each of the resulting pellets are resuspended in 10 mL of cold (4° C.) membrane buffer (25 mM HEPES, pH 7.4, 140 mM NaCl, 1.2 mM MgCl₂, 2.5 mM CaCl₂, 10 μG/mL Aprotinin, 10 μG/mL Leupeptin) and Dounce homogenized using 10-12 strokes. Dilute to 30 mL with buffer and centrifuge at 18,000 rpm, 4° C., 15 minutes (Sorvall RC5C Centrifuge). The resulting pellet is resuspended in cold membrane buffer in a total of ¼ of the original volume by vortexing and aspiration using a syringe and 27 gauge needle.

Protein content is determined (Bradford, Bio-Rad Protein Assay). Membranes are aliquoted in microcentrifuge tubes and quick frozen in liquid nitrogen. Store at −80° C. until use.

The membrane binding buffer is composed of 25 mM HEPES, pH 7.4, 140 mM NaCl, 1.2 mM MgCl₂, 2.5 mM CaCl₂, 0.1% BSA. 160 μL of membrane binding buffer containing 0.5 μg membrane protein is added to 20 μL of 1.0 nM [¹²⁵I]-NDP-α-MSH (final concentration is 0.1 nM) and 20 μL of competing drug or buffer and incubated for 90 minutes at 37° C.

The mixture is filtered with Brandel Microplate 96 filter apparatus using 96-well GF/B filter presoaked in 1-% polyethyleneimine (Sigma). The filter is washed (4 times with a total of 1 mL per well) with cold wash buffer consisting of 20 mM HEPES, pH 7.4, 5 mM MgCl₂.

The filter is dried and punched into a 96 well sample plate (Wallac, 1450-401). 100 μl of Wallac Optiphase Supermix scintillation fluid is added to each well. The top is sealed and the plates are shaken to insure that the filters are thoroughly soaked with fluid. Plates are then counted in a Wallac Microbeta Trilux Scintillation and Luminescence Counter (Model 1450). Dose-response curves are fitted by linear regression analyses and IC₅₀ values are calculated using ExcelFit.

B. Functional Assay

Functional membrane based [³⁵S]GTPγS binding assays are developed to discriminate agonists and antagonists.

Membrane preparation. Cells (HEK-293 cells expressing the human MC4R) are grown in Minimum Essential Medium with Earle's salts and L-glutamate (Life Technologies, Cat. #11095-080) containing 10% heat-inactivated fetal bovine serum, 400 μg/mL geneticin and 100 mM sodium pyruvate in T175 flasks. Upon reaching confluence, cells are dissociated from tissue culture flasks by rinsing with Ca²⁺ and Mg²⁺ free phosphate buffered saline (Life Technologies, Cat. #14190-144) and detached following 5 minutes incubation at 37° C. with enzyme free cell dissociation buffer (Life Technologies, Cat. #13151-014). Cells are collected by centrifugation and resuspended in membrane preparation buffer consisting of 20 mM HEPES, pH 7.4,10 mM EDTA, 10 μg/mL aprotinin and 10 μg/mL leupeptin. The suspension is homogenized by polytron PT3000 for 30 sec at 20,000 rpm, and centrifuged at 35,000×g for 15 minutes at 4° C. The pellet is resuspended in membrane preparation buffer and the last centrifugation is repeated. The final pellet is resuspended in membrane storage buffer consisting of 20 mM HEPES, pH 7.4, 0.1 mM EDTA, 10 μg/mL aprotinin and 10 μg/mL leupeptin. Protein concentration is determined by the Bio-Rad method (Bio-Rad, Cat.#500-0006) and the preparation is diluted to a final protein concentration of 1 mg/mL. Aliquots are stored at −70° C. until used.

[³⁵S]GTPγS membrane binding assay. Compounds are dissolved at 10 mM concentration in DMSO and diluted to the requited concentration into assay buffer. GTPγS to determine nonspecific binding is prepared at 100 μM concentration in assay buffer. The final concentration of DMSO in the assay is 1%. The assay buffer is consisting of 20 mM HEPES, pH 7.4,100 mM NaCl, 5 mM MgCl₂, 0.5 μM GDP, 10 μg/mL saponin, 10 μg/mL aprotinin and 10 μg/mL leupeptin. The assay is composed by adding 50 μL 10×drug solution, 200 μL membrane preparation (containing 2-4 μg protein), 50 μL [³⁵S]GTPγS (100,000-150,000 CPM) and 200 μL assay buffer to achieve a total volume of 500 μL. The assay mixture is incubated at room temperature for exactly 30 minutes. The reaction is terminated by rapid filtration under vacuum through Whatman GF/B filters using a Brandel 96 wells cell harvester, followed by washing four times with cold wash buffer consisting of 20 mM HEPES, pH 7.4, and 5 mM MgCl₂. The filters are air-dried and 200 μL Wallac, Optiphase Super Mix, liquid scintillation cocktail is added to each filter. The bound radioactivity (CPM) is determined by Wallac Trilux 1450 MicroBeta liquid scintillation and Luminescence counter after six hours.

Data interpretation. NDP-α-MSH is used as reference compound and its maximal stimulation is measured at 1 μM (Ref CPM 100%). Total drug-independent binding (Total CPM) is measured in the absence of compounds. Response triggered by compounds is expressed as percent NDP-α-MSH response. Compound dose response curves are generated by Excel XL Fit. The top of the curve represents the compound's intrinsic activity expressed as % of maximal stimulation.

C. Radioligand Binding Assays

Binding of [¹²⁵I]-(Nle⁴, D-Phe⁷)-α-MSH to human melanocortin receptors was performed using membrane homogenates from Hi5 cells that express recombinant MC4 receptors (Hi5-MC4 cells) and from HEK-293 cells that express recombinant MC3 receptors (HEK-MC3 cells) or MC5 receptors (HEK-MC5 cells) as well as from HBL cells expressing the human MC1R receptor. Homogenates (˜0.5 μg protein/well) were incubated with [¹²⁵I]-(Nle⁴,D-Phe⁷)-α-MSH (100 pM for assays with MC4 receptors and 50 pM for assays with MC3/5 receptors) and increasing concentrations of competitors (final concentration of DMSO=1%) for 90 min at 37° C. in buffer consisting of 25 mM HEPES (pH 7.4), 140 mM NaCl, 2.5 mM CaCl₂, 1.2 mM MgCl₂ and 0.1% BSA (10 μg/ml aprotinin and 10 μg/ml leupeptin were added to assays with MC3/5 receptors). Assays were stopped by addition of cold wash buffer (20 mM HEPES and 5 mM MgCl₂ for assays with MC4 receptors and 20 mM HEPES for assays with MC3/5 receptors). Filtration over glass fiber filters (Whatman GF/B previously soaked in 1% PEI for assays with MC4 receptors or 0.5% PEI for assays with MC3/5 receptors) was performed using a Brandel cell harvester. Non-specific binding was defined with 1 μM NDP-α-MSH.

The following Examples illustrate embodiments of the inventive combination, exemplary MC-1R agonists, MC-4R agonists, and starting materials for making those compounds, and are not intended to limit the scope of the claims. For ease of reference, the following abbreviations are used herein:

### Abbreviations

Boc=tert-butoxycarbonyl

CBZ=benzyloxycarbonyl

DEA=diethylamine

DMAP=4-dimethylaminopyridine

DMF=N,N-dimethylformamide

DMSO=dimethylsulfoxide

EDC=3-ethyl-3′-(dimethylamino)propyl-carbodiimide hydrochloride

Et=ethyl

EtOH=ethanol

EtOAc=ethyl acetate

FMOC=fluorenylmethoxycarbonyl

HOBT=1-hydroxybenzotriazole hydrate

NMM=N-methylmorpholine

Me=methyl

MeOH=methanol

mp=melting point

THF=tetrahydrofuran

TFA=trifluoroacetic acid

tic=thin layer chromatography

RT=room temperature

h=hours

HCl=hydrogen chloride

mmol=millimole

Et₃N=triethylamine

EtOAc=ethyl acetate

Et₂O=diethyl ether

Na₂SO₄=sodium sulfate

NaOH=sodium hydroxide

LiOH=lithium hydroxide

CH₂Cl₂=methylene chloride

HPLC=high pressure liquid chromatography

LRMS=low resolution mass spectrometry

In the examples, when a letter is used in a parenthetical or superscript following the term HPLC, MS, or HPLC/MS, as in “HPLC/MS (A)”, “LC/MS (B)”, MS Data^{a}, or following the data, such as 3.28^{a}, the letter denotes the conditions used for the HLPC/MS, as follows:

Method A: Column Primesphere C18-HC 4.6×30 mm, gradient time: 2 min., Hold time: 1 min., Flow rate: 4 mL/ min, Detector Wavelength: 220 nM, Solvent A=10% AcCN/90% H₂O/5 mM NH₄OAc, Solvent B=90% ACCN/10% H₂O/5 mM NH₄OAc, Start % B=0/Finish % B=100;

Method B: Column Primesphere C18-HC 4.6×30 mm, gradient time: 2 min., Hold time: 1 min., Flow rate: 4 mL/min, Detector Wavelength: 220 nM, Solvent A: 10% AcCN/90% H₂O/0.1% TFA, Solvent B: 90% AcCN/10% H₂O/0.1% TFA, Start % B=0/Finish % B=100;

Method C: Column Primesphere C18-HC 4.6×30 mm, gradient time: 3 min., Hold time: 1 min., Flow rate: 4 mL min, Detector Wavelength: 220 nM, Solvent A: 10% AcCN/90% H₂O /0.1% TFA, Solvent B: 90% AcCN/10% H₂O/0.1% TFA, Start % B=0/Finish % B=100, Detector Wavelength: 220 nM;

Method D: Column: Premisphere 5μ-C8 21×100 mm, acetonitrile-5 mM NH₄OAc/water: 7 min. gradient from 20% AcCN to 90% AcCN at 220 nm. Flow rate: 20 mL/min.);

Method E: Column: YMC ODS-A C18 4.6×150 mm; Flow rate: 1 mL min, Solvent system: 0-100% B in 30 min. Solvent A: 10% CH₃CN-90% H₂O-5 mM NH₄OAc; Solvent B: 90% CH₃CN-10% H₂O-5 mM NH₄OAc; UV: 220 nm;

Method F: Column: Combiscreen C8 S-5 4.6×50 mm; Flow rate: 4 mL/min, Solvent system: 0-100% B in 2 min. Solvent A: 10% CH₃CN-90% H₂O-5 mM NH₄OAc; Solvent B: 90% CH₃CN-10% H₂O-5 mM NH₄OAc; UV: 220 nm;

Method G: Column: Combiscreen C8 S-5 4.6×50 mm; Flow rate: 4 mL/min, Solvent system: 0-100% B in 4 min. Solvent A: 10% CH₃CN -90% H₂O-0.1% TFA; Solvent B: 90% CH₃CN -10% H₂O-0.1% TFA; UV: 220 nm;

Method H: Column: YMC ODS-A C18 4.6×150 mm; Flow rate: 1mL/min, Solvent system: 30-100% B in 30 min. Solvent A: 10% CH₃CN -90% H₂O-0.1% TFA; Solvent B: 90% CH₃CN-10% H₂O-0.1% TFA; UV: 220 nm;

Method I: Assignation from another HPLC analysis (with 0.1% TFA);

Method J: Column: Premisphere-5u C8 4.6×30 mm; Flow rate: 4 mL/min, Solvent system: 0-100% (90% CH₃CN -10% H₂O-5 mM NH₄OAc), 2 min. gradient; UV: 220 nm; p0 Method K: Column: YMC S5 C18 4.6×150 mm, Flow rate: 1 mL/min, Solvent system: 0-100% (90% CH₃CN-10% H₂O-5 mM NH₄OAc), 30 min. gradient; UV: 220 nm;

Method L: Column: Xterra-C8 4.6×30 mm; Flow rate: 4 mL/min, Solvent system: 0-100% B in 2 min. Solvent A: 10% CH₃CN-90% H₂O-5 mM NH₄OAc; Solvent B: 90% CH₃CN-10% H₂O-5 mM NH₄OAc; UV: 220 nm;

Method M: Column: YMC-Pack S5 Phenyl 4.6×50 mm; Flow rate: 3 mL/min, Solvent system: 0-100% B in 2 min. Solvent A: 10% CH₃CN-90% H₂O-0.05% TFA; Solvent B: 90% CH₃CN-10% H₂O-0.05% TFA; UV: 220 nm.

### Examples of Co-Administration

### Combination of MC-1R Agonist and cAMP-PDE Inhibitor

Melanocortin receptor agonists and cAMP-PDE inhibitors were evaluated for anti-inflammatory activity in vivo by using an endotoxin-induced TNF-α accumulation model in Balb/C mice. Compounds were administered either by s.c injection or co-injected with endotoxin in the tail vein. Both rolipram and melanocortin agonists inhibit endotoxin-induced TNF-α accumulation in this model.

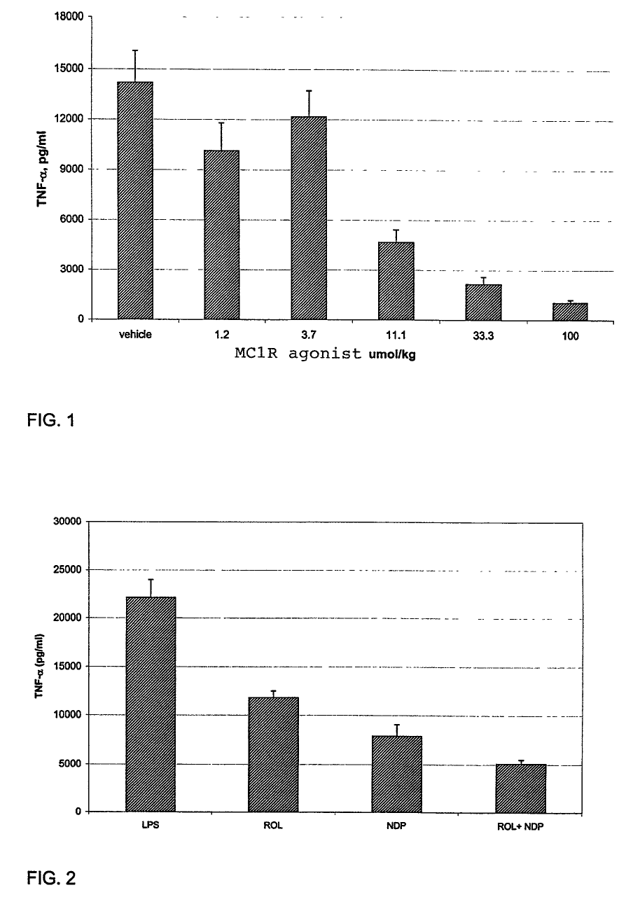

FIG. 1 reports the results of administration of the compound of Example 11, below, in this model. Example 11 was administered by s.c. injection to five mice for each dose of 1.2 μmol/kg, 3.7 μmol/kg, 11.1 μmol/kg, 33.3 μmol/kg, and 100 μmol/kg. The compound was administered 1 hour prior to the LPS-induced challenge. FIG. 1 reports the inhibition in endotoxin-induced TNF-α production, showing a dose dependent response. 67% inhibition was observed at 11.1 umol/kg, and a maximal inhibition of 92% was observed at the highest dose (100 μmol/kg). The compound of Example 11 is a highly selective agonist of the MC-1R with a potency of about 20 nM.

A second experiment was conducted in which the effect of the cAMP-PDE inhibitor rolipram and the melanocortin receptor agonist NDP-MSH was determined. The results are shown in FIG. 2 and summarized in Table 1, below.

TABLE 1

| | % Inhibition |
|---|---|
| Rolipram | 47 |
| NDP-MSH | 64 |
| NDP + Rolipram | 77 |

As shown in FIG. 2 and Table 1, administration of NDP-MSH in combination with rolipram provides surprisingly enhanced therapeutic benefits over administration of either the MC-1R agonist or rolipram alone. The four bar graphs of FIG. 2 reflect the inhibition in LPS-induced TNF-α a production in balb/C mice upon administration of (1) no agent (control); (2) 10 ug/kg ROLIPRAM (3) 2.5 mg/kg NDP-MSH and (4) 10 ug/kg ROLIPRAM in combination with 2.5 mg/kg NDP-MSH. Compounds were co-administered with LPS by tail vein injection. As can be seen, administration of rolipram alone (10 ug/kg) resulted in a 47% inhibition of LPS-induced TNF-α levels. Administration of 2.5 mg/kg NDP-MSH inhibited TNF-α levels by 64%. Co-administration of both agents resulted in a substantially increased inhibition of 77%. As can be seen, the combination of melanocortin receptor agonist and PDE-4 inhibitor produced an additive effect in that inhibition achieved with NDP-MSH and rolipram was greater than achieved with either agent alone.

### Examples of Melancortin Receptor Agonists (MC-R Agonists) for use with cAMP-PDE Inhibitors

### Step A

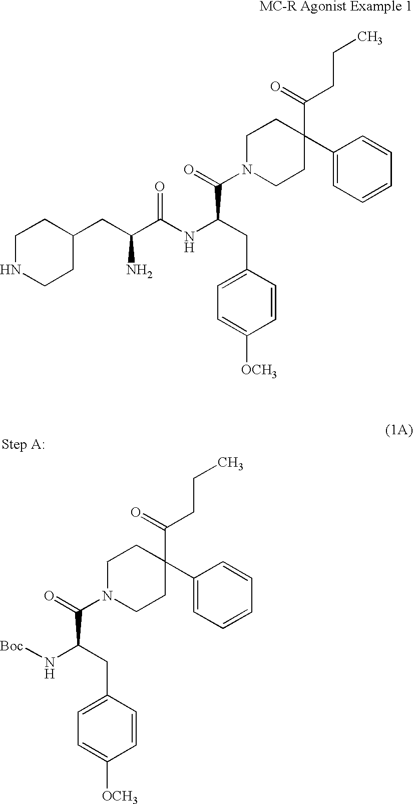

To a solution of N-Boc-D-4-methyltyrosine

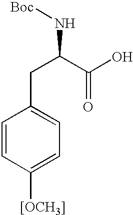

(4.9 g, 16.5 mmol), EDC (4.3 g, 22.5 mmol), HOBT (3.0 g, 22.5 mmol), DMAP (0.2 g, 0. 15 mmol) in CH₂Cl₂, and DMF (1:1, 50 mL) were added Et₃N (10.5 mL, 75.0 mmol) and 4-butanoyl-4-phenyl-piperdine hydrogen chloride

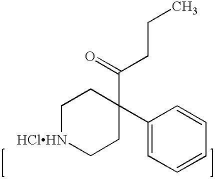

(4.0 g, 15.0 mmol), sequentially. The reaction mixture was stirred at RT overnight. The reaction mixture was diluted with EtOAc (200 mL) and washed with HCl (1 N, 200 mL), water (200 mL), NaOH (0.5 N, 200 mL), and water (200 mL). The organic layer was dried over anhydrous Na₂SO₄, and the solvent was subsequently removed under reduced pressure. The resulting material was >90% pure as judged by HPLC and used without further purification.

### Step B

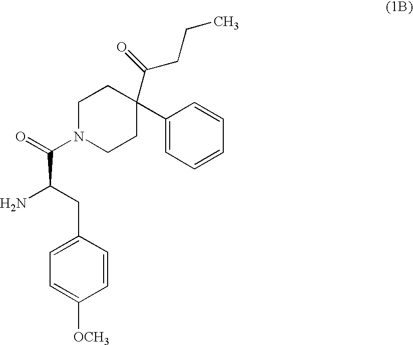

To a solution of compound 1A (12.0 mmol) in wet CH₂Cl₂ (30 mL plus 2 mL water) was added TFA (15 mL). The solution was stirred at RT for 1 h before removing the solvents. The residue was dissolved in EtOAc (300 mL) and washed with water (200 mL), NaOH (0.5 N, 200 mL), and water (200 mL). The organic layer was dried over anhydrous Na₂SO₄, and the solvent removed under reduced pressure. The resulting material (compound 1B) was >90% pure as judged by HPLC and used without further purification.

### Step C

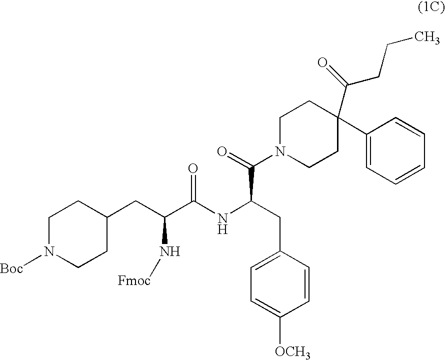

To a solution of Nα-Fmoc-3-(4-N-Boc-piperidine)-L-alanine (0.33 g, 0.67 mmol), EDC (0.18 g, 0.92 mmol), HOBT (0.09 g, 0.92 mmol), DMAP (catalytic) in CH₂Cl₂, and DMF (1:1, 50 mL) were added Et₃N (0.25 mL, 1.8 mmol) and compound 1B (0.25 g, 0.61 mmol), sequentially. The reaction mixture was stirred at RT overnight. The reaction mixture was diluted with EtOAc (200 mL) and washed with HCl (1 N, 200 mL), water (200 mL), NaOH (0.5 N, 200 mL), and water (200 mL). The organic layer was dried over anhydrous Na₂SO₄, and the solvent was subsequently removed under reduced pressure to provide compound 1C.

### Step D

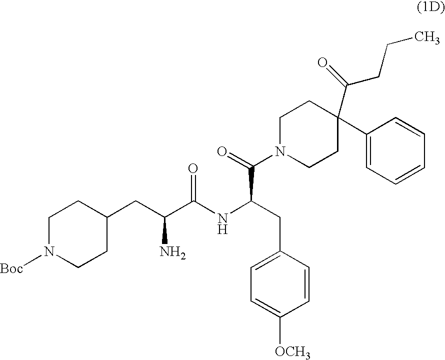

Compound 1C was treated with diethylamine in CH₂Cl₂ (20%) followed by evaporation, to provide compound 1D.

### Step E

Compound 1D was treated with TFA as described in Step B. Example 1 was obtained which was purified by preparative HPLC with a purity of 89% as judged by HPLC.

### MC-R Agonist Example 2

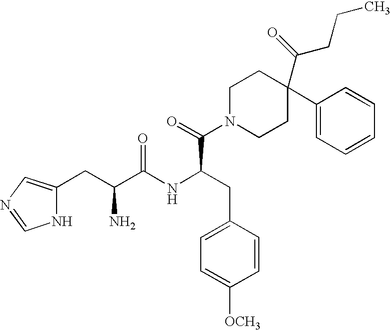

### Step A

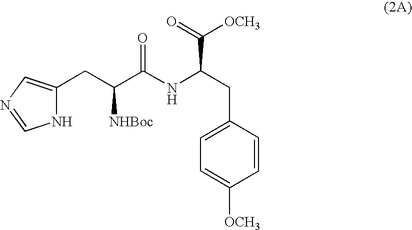

To a solution of N-Boc-L-histidine

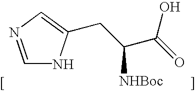

(3.1 g, 12.7 mmols), EDC (3.6 g, 19.1 mmols), HOBT (2.6 g, 19.1 mmols), DMAP (0.16 g, 1.3 mmols) in CH₂Cl₂, and DMF (1:1, 50 mL) were added Et₃N (8.8 mL, 64.0 mmols) and D-4-methoxyphenylalanine methyl ester hydrochloride

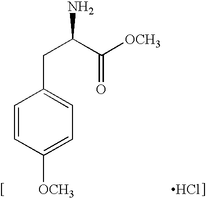

(2.9 g, 12.0 mmol), sequentially. The reaction mixture was stirred at RT overnight. The reaction mixture was diluted with EtOAc (200 mL) and washed with water (200 mL), NaOH (0.5 N, 200 mL), and water (200 mL). The organic layer was dried over anhydrous Na₂SO₄, and the solvent was subsequently removed under reduced pressure. The resulting compound 1A was >90% pure as judged by HPLC and used without further purification in Step B.

### Step B

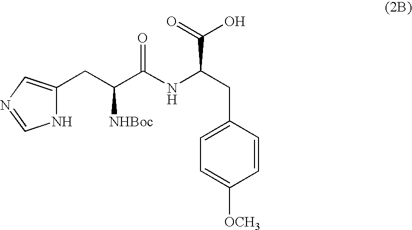

To a solution of Compound 2A (12.0 mmol) in CH₃OH (13 mL) was added NaOH (2N, 13 mL) to make the final concentration of NaOH ˜1 N. This solution was stirred at RT for 2 h before being diluted with water (100 mL). The aqueous layer was extracted with Et₂O (100 mL×2), and the organic matter was discarded. The aqueous layer was acidified with HCl (6 N) to pH ˜2, and extracted with EtOAc (100 mL×2). The combined organic layers were dried over anhydrous Na₂SO₄, and the solvent was subsequently removed under reduced pressure. The resulting Compound 2B was a white solid with a purity >90% as judged by HPLC. This intermediate was used without further purification for Step C.

### Step C

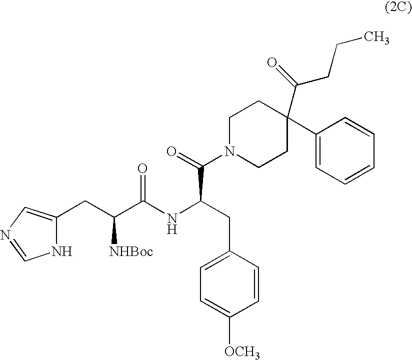

To a solution of Compound 2B (0.5 g, 1.1 mmols), EDC (0.3 g g, 1.6 mmols), HOBT (0.22 g, 1.6 mmols), and DMAP (0.13 g, 1.1 mmols) in CH₂Cl₂ (25 mL) were added Et₃N (0.8 mL, 5.5 mmols) and 4-butyryl-4-phenyl-piperidine hydrochloride (0.35 g, 1.3 mmols), sequentially. The reaction mixture was stirred at RT overnight. The reaction mixture was diluted with EtOAc (100 mL) and washed with HCl (0.5 N, 100 mL), water (100 mL), NaOH (0.5 N, 100 mL), and water (100 mL). The organic layer was dried over anhydrous Na₂SO₄, and the solvent removed under reduced pressure. The resulting Compound 1C was >90% pure as judged by HPLC and used without further purification in Step D.

### Step D

To a solution of the Boc-protected Compound 2C (1.1 mmols) in wet CH₂Cl₂ (20 mL plus 1 mL water) was added TFA (10 mL). The solution was stirred at RT for 1 h before the solvents were removed. The crude reaction mixture was purified by preparative HPLC to obtain compound 2D at >95% purity as judged by HPLC. HPLC (min)=2.5, MS (M+H)⁺=546.4.

### MC-R Agonist Example 3

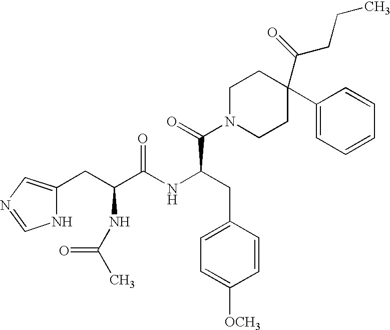

To a solution of Example 2 (0.1 g, 0.18 mmol) in CH₂Cl₂ (10 mL) was added Et₃N (0.075 mL, 0.54 mmol). This solution was cooled to 0° C., and then acetyl chloride was added (0.02 g, 0.27 mmol). The reaction mixture was stirred at RT until all the amine was consumed. The reaction mixture was diluted with EtOAc (100 mL) and washed with HCl (0.5 N, 100 mL), water (100 mL), NaOH (0.5 N, 100 mL), and water (100 mL). The organic layer was dried over anhydrous Na₂SO₄, and the solvent removed under reduced pressure to provide Example 3 which was purified by preparative HPLC. Purity=94%, HPLC ret. time (min.)=2.71, MS (M+H)⁺=588.

### MC-R Agonist Examples 4-26

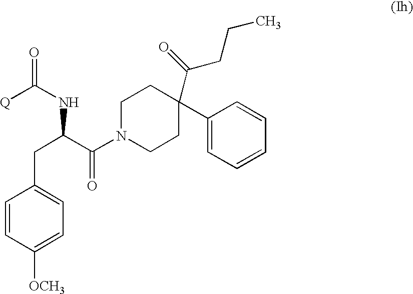

Compounds of formula (Ih), above, wherein the group Q has the values listed in Table 2, were prepared following the same or similar procedures described above for Examples 1 and 2.

TABLE 2

| | | | HPLC RT | Mass |
|---|---|---|---|---|
| Ex No. | Q | Purity (%) | (min) | (M + H) |
| 4 | 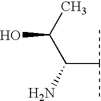 | 90 | 3.2 | 510.45 |
| 5 | 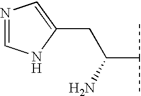 | 91 | 2.5 | 546.36 |
| 6 | 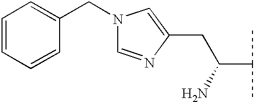 | 95 | 2.7 | 636.21 |
| 7 | 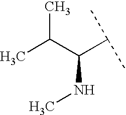 | 92 | 3.4 | 522.5 |
| 8 | 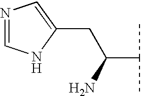 | 93 | 2.5 | 546.4 |
| 9 | 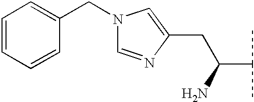 | 90 | 2.8 | 636.26 |
| 10 | 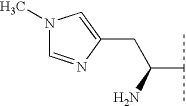 | 92 | 2.5 | 560.39 |
| 11 | 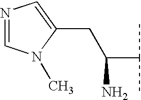 | 94 | 2.5 | 560.15 |
| 12 | 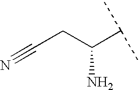 | 93 | 3.0 | 505.35 |
| 13 | 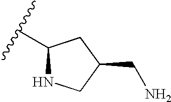 | 92 | 2.5 | 535 |
| 14 | 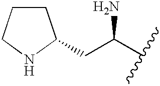 | 91 | 2.5 | 549 |
| 15 | 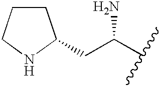 | 86 | 2.56 | 549.31 |
| 16 | 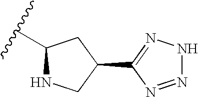 | 85 | 2.72 | 574 |
| 17 | 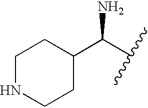 | 88 | 2.49 | 549.3 |
| 18 | 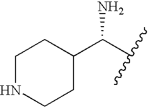 | 91 | 2.52 | 549.31 |
| 19 | 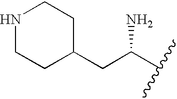 | 89 | 2.53 | 563.42 |
| 20 | 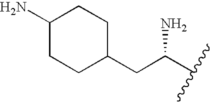 | 92 | 2.58 | 577.38 |
| 21 | 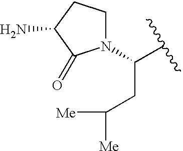 | 82 | 3.07 | 605 |
| 22 | 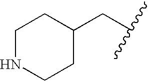 | 91 | 2.76 | 534.33 |
| 23 | 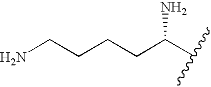 | 92 | 2.5 | 537.44 |
| 24 | 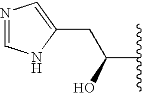 | 93 | 2.71 | 547.28 |
| 25 | 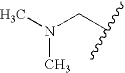 | 93 | 2.71 | 494.31 |
| 26 | 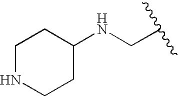 | 90 | 2.52 | 549.32 |

### MC-R Agonist Examples 27-30

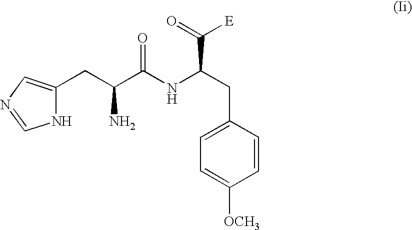

Compounds of formula (Ii), above, wherein the group E is as shown in Table prepared following the same or similar procedures described above for Example 2.

TABLE 3

| | | | HPLC RT | Mass |
|---|---|---|---|---|
| Ex. No. | E | Purity (%) | (min) | (M + H) |
| 27 | 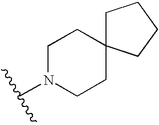 | 95 | 2.43 | 454 |
| 28 | 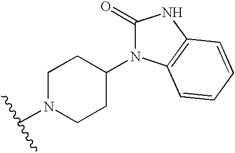 | 98 | 1.96 | 532 |
| 29 | 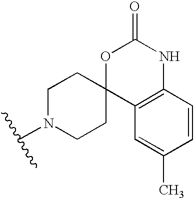 | 95 | 2.11 | 547 |
| 30 | 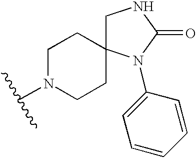 | 90 | 2.03 | 546 |

### MC-R Agonist Examples 31-33

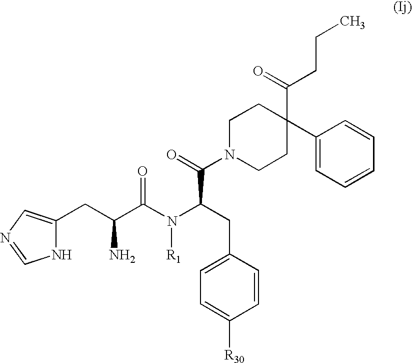

Compounds of formula (Ij), above, wherein the groups R₁ and R₃₀ have the values listed in Table 4, were prepared following the same procedures as in Example 2.

TABLE 4

| | | | Purity | HPLC | Mass |
|---|---|---|---|---|---|
| Ex. No. | R₁ | R₃₀ | (%) | RT (min) | (M + H) |
| 31 | H | Cl | 90 | 3.08 | 551 |
| 32 | H | H | 86 | 2.88 | 515 |
| 33 | CH₃ | Cl | 85 | 3.26 | 565 |

### MC-R Agonist Examples 34-39

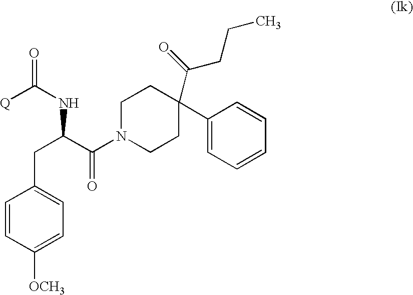

Compounds of formula (Ik), wherein the group Q has the values listed in Table 5, were prepared following the procedure described above for Examples 2-3.

TABLE 5

| Example | | Purity | HPLC RT | Mass |
|---|---|---|---|---|
| No. | Q | (%) | (min) | (M + H) |
| 34 | 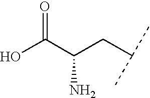 | 85 | 2.7 | 524.22 |
| 35 | 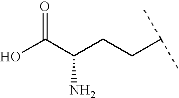 | 84 | 2.7 | 538.24 |
| 36 | 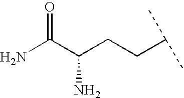 | 100 | 2.7 | 537.28 |
| 37 | 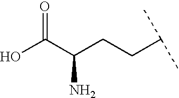 | 81 | 2.7 | 538.14 |
| 38 | 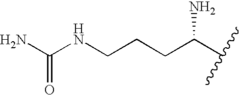 | 83 | 2.67 | 566.31 |
| 39 | 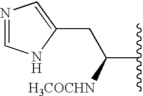 | 94 | 2.71 | 588 |

### MC-R Agonist Examples 40-51

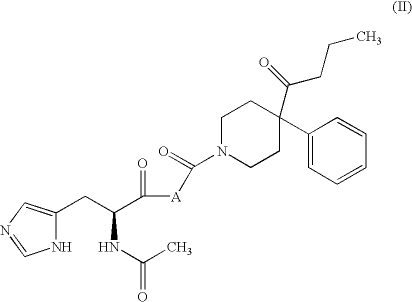

Compounds of formula (II), above, wherein A has the values listed in Table 6, were prepared following the same procedure as described above for Examples 2-3.

TABLE 6

| Example | | Purity | HPLC RT | Mass |
|---|---|---|---|---|
| No. | A | (%) | (min) | (M + H) |
| 40 | —CH₂CH₂— | 76 | 2.7 | 482.32 |
| 41 | 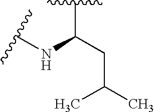 | 79 | 3.2 | 524.39 |
| 42 | 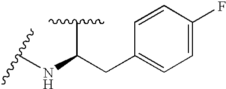 | 85 | 3.3 | 576.31 |
| 43 | 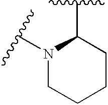 | 73 | 3.0 | 522.38 |
| 44 | 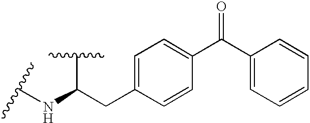 | 71 | 3.4 | 662.34 |
| 45 | 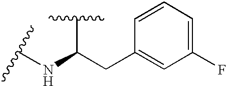 | 84 | 3.3 | 576.31 |
| 46 | 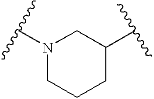 | 87 | 2.9 | 522.38 |
| 47 | 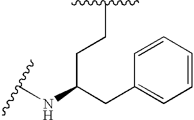 | 76 | 3.3 | 586.36 |
| 48 | 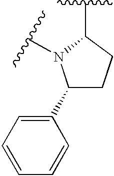 | 80 | 3.2 | 584.34 |
| 49 | 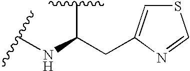 | 82 | 2.9 | 565.27 |
| 50 | 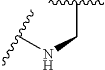 | 81 | 2.7 | 468.29 |
| 50 | 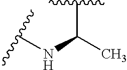 | 84 | 2.8 | 482.31 |
| 51 | 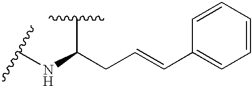 | 75 | 3.4 | 584.33 |

### MC-R Agonist Examples 52-53

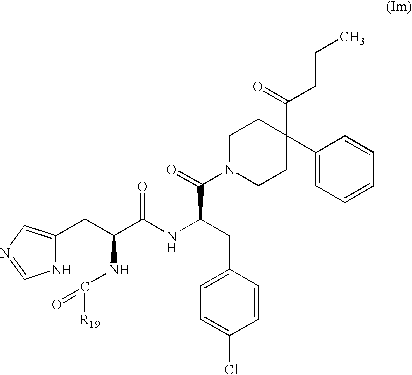

Compounds of formula (Im), above, wherein the group R₁₉ has the values listed in Table 7, were prepared following the same or similar procedures described above for Examples 2-3.

TABLE 7

| | | | HPLC | |
|---|---|---|---|---|
| | | Purity | RT | Mass |
| Ex. No. | R₁₉ | (%) | (min) | (M + H) |
| 52 | CH₃ | 96 | | 593 |
| 53 | Ph | 90 | | 655 |

### MC-R Agonist Examples 54-68

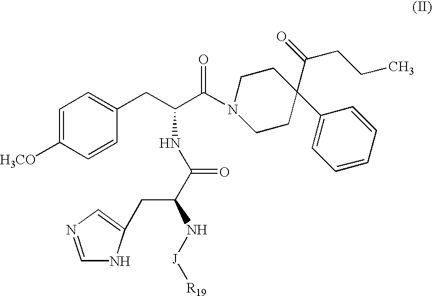

Compounds having the above formula (II), wherein J and R_{·}have the values listed in Table 8, were prepared following the same or similar procedure as for Examples 2-3. For examples 54-58 and 66-68, in the last step, compound 2D was dissolved in DCM and reacted with 1.2 eq of the appropriate sulfonyl chloride or chloroformate in presence of 3 eq of resin bound morpholine (Argonaut Technologies) at RT overnight. After filtration and concentration the residue was purified by RP-prep HPLC. For examples 59-65, in the last step compound 2D was reacted with 1.1 eq of the appropriate isocyanate in toluene at RT overnight. After concentration, the residue was purified by RP-prep HPLC.

TABLE 8

| | | | Purity | HPLC RT | Mass |
|---|---|---|---|---|---|
| Ex. No. | J | R₁₉ | (%) | (min) | (M + H) |
| 54 | —SO₂— | —CH₃ | 86 | 3.03 | 623.8 |
| 55 | —SO₂— | —CH₂CH₃ | 80 | 3.10 | 637.8 |
| 56 | —SO₂— | —CH₂CH₂CH₃ | 95 | 3.20 | 651.8 |
| 57 | —SO₂— | 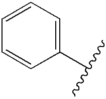 | 95 | 3.22 | 685.8 |
| 58 | —SO₂— | 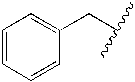 | 95 | 3.32 | 699.9 |
| 59 | —C(═O)NH— | —CH₂CH₃ | 92 | 3.12 | 616.8 |
| 60 | —C(═O)NH— | —CH₂ | 80 | 3.05 | 602.7 |
| 61 | —C(═O)NH— | —CH(CH₃)(CH₃) | 95 | 3.22 | 630.8 |
| 62 | —C(═O)NH— | —CH₂CH₂CH₃ | 95 | 3.23 | 630.8 |
| 63 | —C(═O)NH— | 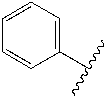 | 95 | 3.36 | 664.8 |
| 64 | —C(═O)NH— | 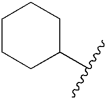 | 95 | 3.46 | 670.9 |
| 65 | —C(═O)NH— | 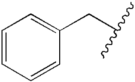 | 95 | 3.36 | 678.8 |
| 66 | —CO₂— | —CH₂CH₂CH₃ | 94 | 3.69 | 631.8 |
| 67 | —CO₂— | —CH₃ | 91 | 3.47 | 603.7 |
| 68 | —CO₂— | —CH₂CH₃ | 90 | 3.56 | 617.8 |

### MC-R Agonist Example 69

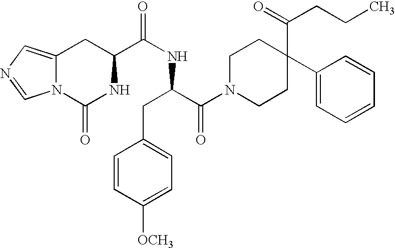

Example 69 was prepared following the same or similar procedure as described above for Example 3. In the last step, compound 3D was reacted with 1.2 eq of phenylchloroformate in DCM in presence of 3 eq of resin bound morpholine. After filtration and concentration, the residue was purified by RP-prep HPLC. Purity=98%, HPLC ret. time (min)=3.42, MS (M+H)⁺=572.

### MC-R Agonist Examples 70-83

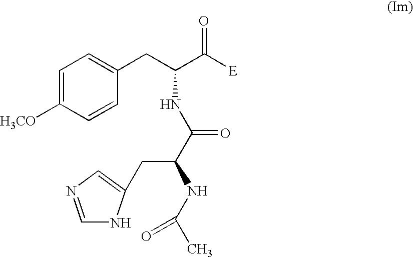

Compounds having the above formula (Im), wherein E has the values listed in Table 9, were prepared following the same or similar procedures as described for Examples 2 and 3.

TABLE 9

| | | Purity | HPLC RT | Mass |
|---|---|---|---|---|
| Ex. No. | E | (%) | (min) | (M + H) |
| 70 | 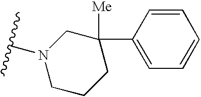 | 88.3% | 3.2 | 531.7 |
| 71 | 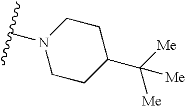 | 82.3% | 3.2 | 497.6 |
| 72 | 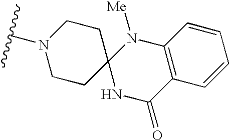 | 71.0% | 2.6 | 587.7 |
| 73 | 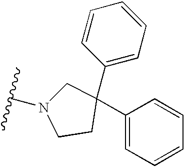 | 93.4% | 3.3 | 579.7 |
| 74 | 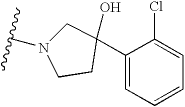 | 79.8% | 3.0 | 554.0 |
| 75 | 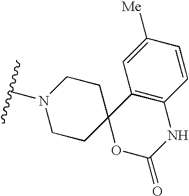 | 82.0% | 2.9 | 588.7 |
| 76 | 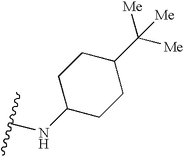 | 90.3% | 3.5 | 511.7 |
| 77 | 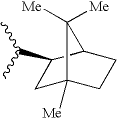 | 88.1% | 3.3 | 509.7 |
| 78 | 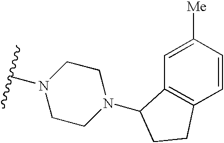 | 87.8% | 2.2 | 588.7 |
| 79 | 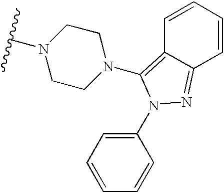 | 89.3% | 3.1 | 634.7 |
| 80 | 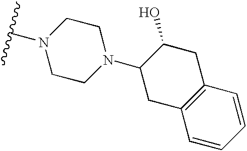 | 90.1% | 2.4 | 588.7 |
| 81 | 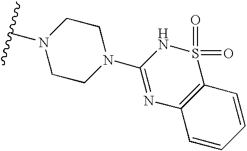 | 88.4% | 2.4 | 622.7 |

### MC-R Agonist Examples 82-86

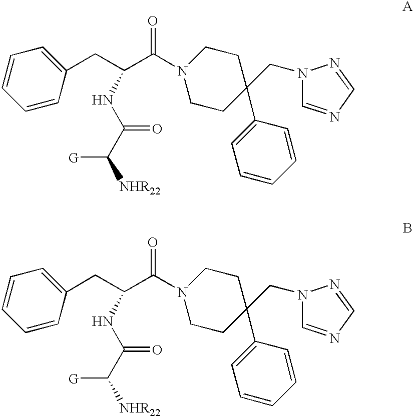

Compounds having the above formulae A or B, wherein G and R₂₂ have the values listed in Table 10, were prepared following the same or similar procedures as described in Example 1.

TABLE 10

| Ex. | | | | Purity | HPLC RT | Mass |
|---|---|---|---|---|---|---|
| No. | Core | G | R₂₂ | (%) | (min) | (M + H) |
| 82 | A | 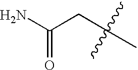 | 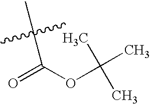 | 82.0% | 3.8 | 644.44 |
| 83 | A | 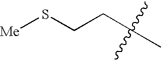 | 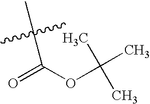 | 80.0% | 4.1 | 661.43 |
| 84 | B | 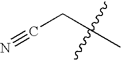 | 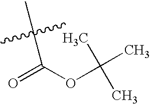 | 91.0% | 3.9 | 626.42 |
| 85 | A | 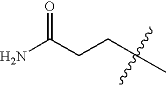 | 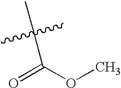 | 89.0% | 3.4 | 600.41 |
| 86 | B | 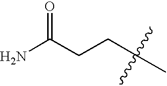 | 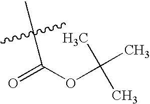 | 94.1% | 3.8 | 658.44 |

### MC-R Agonist Example 87

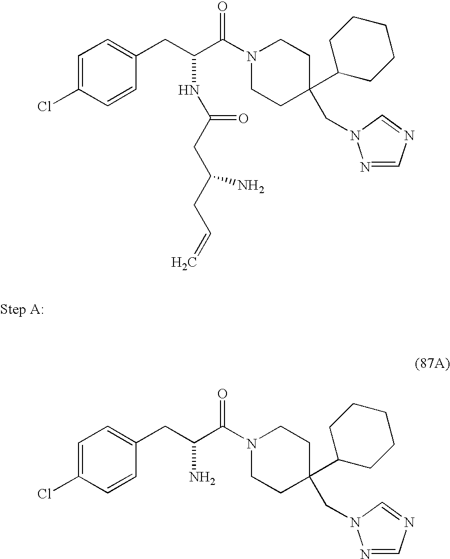

Compound 87A was prepared by coupling of commercially available N-BOC D-4-chlorophenylalanine and 4-Cyclohexyl-4-[1,2,4]triazol-1-ylmethyl-piperidine, followed by deprotection of the BOC group, as described in WO 00/74679.

### Step B

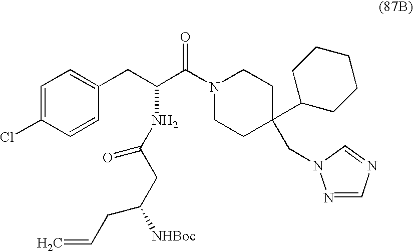

To a solution of the compound 87A and the amino acid having the formula,

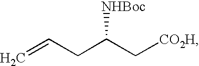

in DCM (12 mL) was added 1-ethyl-3-(3-dimethylaminopropyl) carbodiimide (736 mg, 3.8 mmol) and HOBt (518 mg, 3.8 mmol) at RT. The mixture was stirred at RT overnight and a sat'd solution of ammonium chloride (15 mL) was added. The separated aqueous layer was extracted with DCM (3×25 mL), and the combined organic layers were dried (MgSO₄ anh.), filtered, and evaporated to afford compound 87B which was used in the next step without purification. HPLC (Column: Combiscreen C8 S-5 4.6×50 mm; Flow rate: 4 mL min, Solvent system: 0-100% B in 4 min. Solvent A: 10% CH₃CN-90% H₂O-0.1% TFA; Solvent B: 90% CH₃CN-10% H₂O-0.1% TFA; UV: 220 nm): retention time 2.40 min, purity 99.2%; HPLC (Column: Luna CN 4.6×30 mm; Flow rate: 4 mL min, Solvent system: 0-100% B in 4 min. Solvent A: 10% CH₃CN-90% H₂O-5 mM NH₄OAc; Solvent B: 90% CH₃CN-10% H₂O-5 mM NH₄OAc; UV: 220 nm): retention time 3.06 min, purity 100%; HPLC/MS (Column: YMC ODS-A C18 4.6×50 mm; Flow rate: 4 mL/min, Solvent system: 0-100% B in 2min. Solvent A: 10% CH₃CN -90% H₂O-5 mM NH₄OAc; Solvent B: 90% CH₃CN-10% H₂O-5 mM NH₄OAc; UV: 220 nm; Micromass ZMD 2000, ESI): retention time 1.81 min, purity 97.8%, MS pos. m/z 541 (M+H)⁺; MS (Finigan TSQ 7000, ESI) m/z541 (M+H)⁺; ¹H NMR (400 MHz, CD₃OD) δ ppm (two rotamers; ratio 1.8:1) 8.45 (1H, s, minor rotamer), 8.43 (1H, s, major rotamer), 7.99 (1H, s, minor rotamer), 7.94 (1H, s, major rotamer), 7.31 (2H, d, J=8 Hz, major rotamer), 7.28 (2H, d, J=8 Hz, minor rotamer), 7.23 (2H, d, J=8 Hz, major rotamer), 7.21 (2H, d, J=8 Hz, minor rotamer), 5.82-5.69 (1H, m), 5.26-5.20 (2H, m), 5.05 (1H, dd, J=6, 12 Hz), 4.26 (2H, s, major rotamer), 4.25 (2H, s, minor rotamer), 3.69-3.58 (1H, m), 3.55-3.43 (2H, m), 3.40-3.32 (1H, m), 3.01-2.84 (2H, m), 2.63-2.55 (1H, m), 2.50-2.43 (1H, m), 2.37-2.30 (2H, m), 1.85-1.63 (6H, m), 1.45-0.86 (8H, m). ¹³C NMR (100.61 MHz, CD₃OD) δ ppm (two rotamers; ratio 1.8:1) 171.7 (s, major rotamer), 171.6 (s, minor rotamer), 171.3 (s), 151.7 (d), 146.4 (d), 136.7 (d, minor rotamer), 136.6 (d, major rotamer), 134.1 (s, major rotamer), 134.0 (d, minor rotamer), 132.8 (s, major rotamer). 132.7 (s, minor rotamer), 2×132.3 (d, major rotamer), 2×132.1 (d, minor rotamer), 2×129.8 (d, major rotamer), 2×129.7 (minor rotamer), 121.0 (t), 53.0 (t, minor rotamer), 52.7 (t, major rotamer), 51.6 (d, minor rotamer) 51.4 (d, major rotamer), 43.0 (d), 42.8 (t, minor rotamer), 42.6 (t, major rotamer), 39.1 (s), 2×38.9 (t, major rotamer), 38.7 (t, major rotamer), 38.3 (t, minor rotamer), 38.0 (s, major rotamer), 37.9 (s, minor rotamer), 37.1 (t, minor rotamer), 37.0 (t, major rotamer), 31.2 (t), 30.6 (t), 2×28.2 (t), 27.6 (t), 3×27.4 (t); ir νₘₐₓ, KBr) cm⁻¹: 3565-2500 (broad), 1683, 1635, 1456, 1203, 1139.

### Step C

### EXAMPLE 87

To a solution of Compound 87B in DCM (10 mL) was added a 20% (v/v) solution of TFA in DCM (1.6 mL) at RT. The mixture was stirred at RT for 8 h and evaporated under reduced pressure. The residue was purified using preparative HPLC and after evaporation, the residue was lyophilized to afford Example 87 as the TFA salt. HPLC ret. time (min)=1.54^{b}, MS (M+H)⁺=541.

### MC-R Agonist Examples 88-92

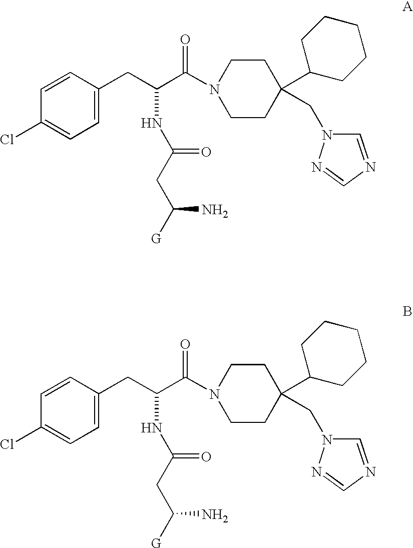

Compounds having the above formulae A or B, wherein G has the values listed in Table 11 were prepared following the same or similar procedure as described for Example 1.

TABLE 11

| | | | HPLC | |
|---|---|---|---|---|
| | | | Retention | |
| Ex. | | | Time | MS Data |
| No. | Core | G | (min) | (M + H)⁺ |
| 88 | B | 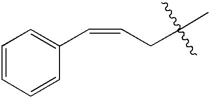 | 3.20^{c} | 617 |
| 89 | A | 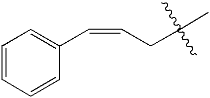 | 3.19^{c} | 617 |
| 90 | A | 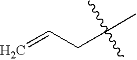 | 1.52^{b} | 541 |
| 91 | A | CH₃SCH₂CH₂— | 1.74^{a} | 575 |
| 92 | B | 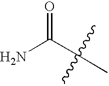 | 1.52^{a} | 544 |

### MC-R Agonist Examples 93-98

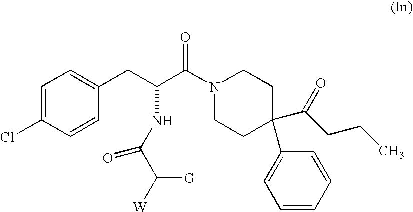

Compounds of formula (In), above, wherein the groups G and W have the values listed in Table 12, were prepared following the same or similar procedure described above for Example 2, using a different amino acid in place N-Boc-L-histidine in Step A.

TABLE 12

| | | | | HPLC | |
|---|---|---|---|---|---|
| Ex | | | Purity | Ret. | Mass |
| No. | G | W | (%) | Time (min) | (M + H) |
| 93 | 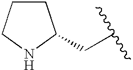 | 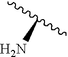 | 91 | 2.5 | 549 |
| 94 | 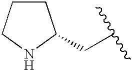 | 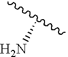 | 86 | 2.56 | 549.31 |
| 95 | 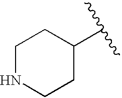 | 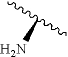 | 88 | 2.49 | 549.3 |
| 96 | 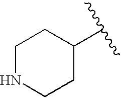 | 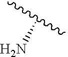 | 91 | 2.52 | 549.31 |
| 97 | 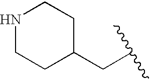 | 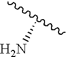 | 89 | 2.53 | 563.42 |
| 98 | 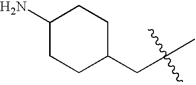 | 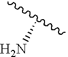 | 92 | 2.58 | 577.38 |

### MC-R Agonist Example 99

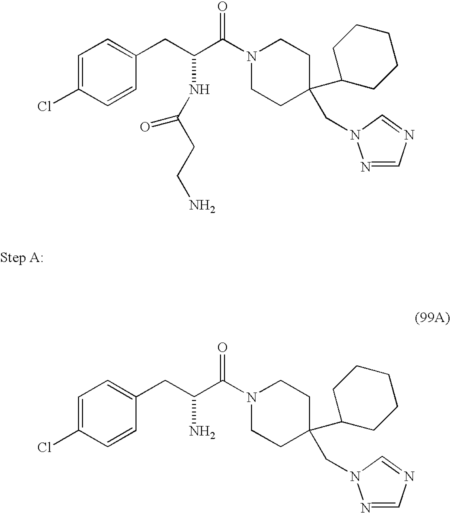

Compound 99A was prepared by coupling of commercially available N-BOC D-4-chlorophenylalanine and 4-Cyclohexyl-4-[1,2,4]triazol-1-ylmethyl-piperidine, followed by deprotection of the BOC group, as described in WO 00/74679, incorporated herein by reference.

### Step B

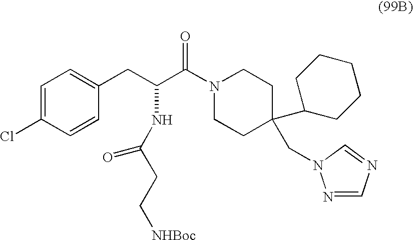

To a solution of α-amino amide from step A (1.1 g, 2.56 mmol) and N-Boc-β-alanine (531 mg, 2.81 mmol) in DCM (12 mL) was added 1-ethyl-3-(3-dimethylaminopropyl) carbodiimide (736 mg, 3.8 mmol) and HOBt (518 mg, 3.8 mmol) at RT. The mixture was stirred at RT overnight and a sat'd solution of ammonium chloride (15 mL) was added. The separated aqueous layer was extracted with DCM (3×25 mL), and the combined organic layers were dried (MgSO₄ anh.), filtered, and evaporated to afford compound 99A which was used in the next step without purification.

### Step C

### EXAMPLE 99

To a solution of Compound 99B (1.0 g, 1.7 mmol) in DCM (10 mL) was added a 20% (v/v) solution of TFA in DCM (1.6 mL) at RT. The mixture was stirred at RT for 8 h and evaporated under reduced pressure. The residue was purified using preparative HPLC and after evaporation, the residue was lyophilized to afford 0.9 g (47% yield) of Example 99 as the TFA salt. HPLC/MS (A), ret. time=1.50 min, purity 86.9%, MS pos. m/z501 (M+H)⁺; HPLC/MS (E), ret. time=10.81 min, purity 100%; ir (νₘₐₓ, KBr) cm⁻¹ 3600-2880,1695,1620; ¹H NMR (400 MHz, MeOH-d₄) δ ppm (two rotamers; 1:2 ratio) 8.43 (1H, s, minor rotamer), 8.42 (1H, s, major rotamer), 7.96 (1H, s, minor rotamer), 7.92 (1H, s, major rotamer), 7.26 (2H, d, J=8.3 Hz, major rotamer), 7.23 (2H, d, J=8.4 Hz, minor rotamer), 7.18 (2H, d, J=8.3 Hz, major rotamer), 7.15 (2H, d, J=8.6 Hz, minor rotamer), 4.98 (1H, t, J=7.8 Hz), 4.21 (2H, s, major rotamer), 4.18 (2H, s, minor rotamer), 3.60 (1H, m), 3.31 (3H, m), 3.08 (2H, m), 2.87 (2H, m), 2.54 (2H, t, J=6.5 Hz), 1.95-0.82 (15H, m). AnaL Calc'd for C₂₆H₃₇ClN₆O₂.3CF₃COOH.2H₂O: C, 43.72; H, 5.04; N, 9.56. Found: C, 43.90; H, 4.31; N, 9.16. AnaL Calc'd for C₂₆H₃₇ClN₆O₂.3HCl.H₂O: C, 49.69; H, 6.74; N, 13.37. Found: C, 49.96; H, 6.75; N, 12.88.

### MC-R Agonist Example 100

### 3-Amino-N-[1-(4-chloro-benzyl)-2-(4-cyclohexyl-4-[1,2,4]triazol-1-ylmethyl-piperidin-1-yl)-2-oxo-ethyl]-2,2-dimethyl-propionamide

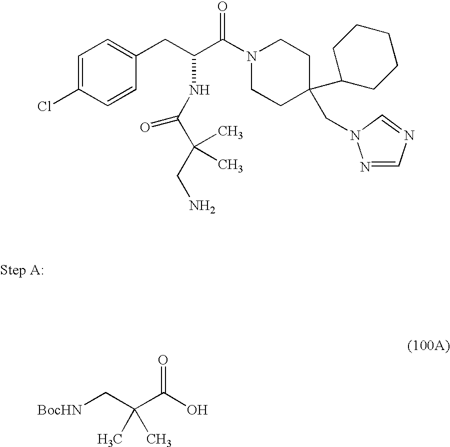

Compound 100A was prepared following the procedure described in Dhokte et al., Tetrahedron Lett., Vol. 39 (1998), at pp. 8771-8774.

### Step B

Example 100 was prepared following the procedure described for the preparation of Example 99, using Compound (100A) in place of Boc-β-alanine in Step A. HPLC/MS (F), ret. time 1.64 min, purity 95.7%, MS pos. m/z 529 (M+H)⁺; HPLC/MS (H), ret. time=12.12 min, purity 95.1%; ¹H NMR (400 MHz, MeOH-d₄) δ ppm (two rotamers; 1:1.4 ratio) 8.56 (1H, s, minor rotamer), 8.53 (1H, s, major rotamer), 8.08 (1H, s, minor rotamer), 8.02 (1H, s, major rotamer), 7.36 (2H, d, J=8.4 Hz, major rotamer), 7.34 (2H, d, J=8.9 Hz, minor rotamer), 7.28 (2H, d, J=8.3 Hz, major rotamer), 7.25 (2H, d, J=8.4 Hz, minor rotamer), 5.07 (1H, m), 4.32 (2H, s), 3.68-3.34 (4H, m), 3.02 (4H, m), 1.98-0.99 (15H, m), 1.34,1.24 (6H, 2s, minor rotamer), 1.33,1.28 (6H, 2s, major rotamer).

### MC-R Agonist Examples 101-108

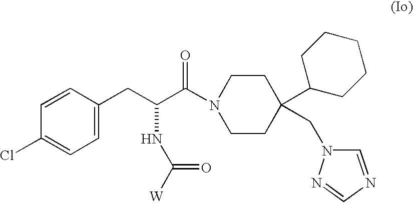

Compounds having the formula (Io), wherein W has the values listed in Table 13, prepared using the same or similar procedure described above for the preparation of Example 100.

TABLE 13

| | | HPLC Ret. | MS Data |
|---|---|---|---|
| Ex. | W | Time (min) | (M + H)⁺ |
| 101 | 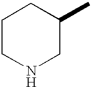 | 1.72^{a} | 541 |
| 102 | 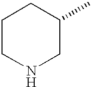 | 1.65^{a} | 541 |
| 102 | 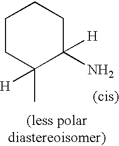 | 1.91^{a} | 555 |
| 104 | 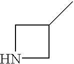 | 1.65^{a} | 513 |
| 105 | 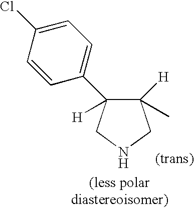 | 1.94^{a} | 637 |
| 106 | 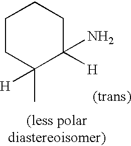 | 1.80^{a} | 555 |
| 107 | CH₃NHCH₂— | 1.83^{a} | 501 |
| 108 | 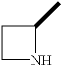 | 1.89^{a} | 513 |
| 109 | 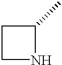 | 1.82^{a} | 513 |
| 110 | (CH₃)₂NCH₂— | 1.84^{a} | 515 |

### MC-R Agonist Example 111

### N-[1-(4-Chloro-benzyl)-2-(4-cyclohexyl-4-[1,2,4]triazol-1-ylmethyl-piperidin-1-yl)-2-oxo-ethyl]-3-dimethylamino-propionamide

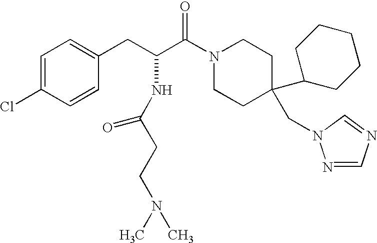

To a vigorously stirred solution of Example 99 (45 mg, 0.09 mmol) and formaldehyde (37% w/w in water, 45 μL, 0.5 mmol) in DCE (1.0 mL) was added sodium triacetoxyborohydride (110 mg, 0.5 mmol) at RT. The mixture was stirred overnight at RT and a sat'd solution of ammonium acetate (5 mL) was added. The separated aqueous layer was extracted with methylene chloride (3×15 mL), and the combined organic layers were dried (Na₂SO₄), filtered and evaporated under reduced pressure. The residue was purified using preparative HPLC and after evaporation, the residue was lyophilized to afford Example 111 as the TFA salt. HPLC/MS (A), ret. time=1.74 min, purity 98.2% Micromass ZMD 2000, ESI): MS pos. m/z 529 (M+H)⁺; HPLC (K), ret. time=19.58 min, purity 84.3%. ¹H NMR (400 MHz, MeOH-d₄), δ ppm (two rotamers; 1:1.7 ratio) 8.56 (1H, s, minor rotamer), 8.53 (1H, s, major rotamer), 8.08 (1H, s, minor rotamer), 8.03 (1H, s, major rotamer), 7.35 (2H, d, J=8.3 Hz, major rotamer), 7.29 (2H, d, J=8.4 Hz, minor rotamer), 7.28 (2H, d, J=8.3 Hz, major rotamer), 7.26 (2H, d, J=8.6 Hz, minor rotamer), 5.00 (1H, m), 4.31 (2H, m), 3.70-2.85 (11H, m), 2.92 (6H, br. s), 2.74 (2H, m), 1.91-0.75 (15H, m).

### MC-R Agonist Example 112

### 3-Acetylamino-N-[1-(4-chloro-benzyl)-2-(4-cyclohexyl-4-[1,2,4]triazol-1-ylmethyl-piperidin-1-yl)-2-oxo-ethyl]-propionamide

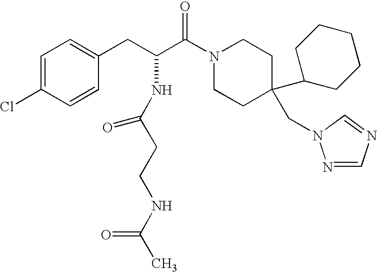

Acetyl chloride (25 μL, 3.3 mmol) was added to a solution of Example 99 (150 mg, 3.0 mmol) and Et₃N (50 μL, 3.6 mmol) in DCM (7 mL) at 0° C. The mixture was stirred at RT overnight and quenched with sat'd ammonium chloride (10 mL). The separated aqueous layer was extracted with methylene chloride (3×15 mL), and the combined organic layers were dried (Na₂SO₄), filtered and evaporated under reduced pressure. The residue was purified using preparative HPLC, and after evaporation, the residue was lyophilized to afford Example 241 as the TFA salt. HPLC/MS (A?), ret. time=1.60 min, purity 91.6%. Micromass ZMD 2000, ESI): MS pos. m/z 543 (M+H)⁺; HPLC (K), ret. time=20.98 min, purity 92.6%. ¹H NMR (400 MHz, MeOH-d₄) δ ppm (two rotamers; 1:1.6 ratio) 8.56 (1H, s), 8.13 (1H, s), 7.35 (2H, d, J=8.1 Hz, major rotamer), 7.30 (2H, d, J=8.1 Hz, minor rotamer), 7.28 (2H, d, J=8.1 Hz, major rotamer), 7.24 (2H, d, J=8.6 Hz, minor rotamer), 5.08 (1H, br. t, J=3.3 Hz), 4.34 (2H, s, major rotamer), 4.29 (2H, s, minor rotamer), 3.70-2.85 (11H, m), 2.74 (2H, m), 1.96 (3H, s, major rotamer), 1.94 (3H, s, minor rotamer), 1.91-0.75 (15H, m).

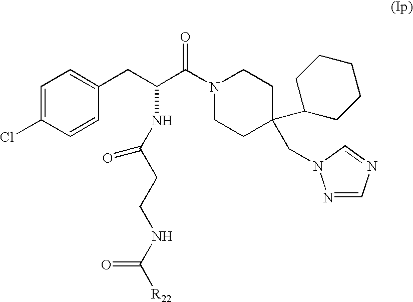

Compounds having the formula (Ip), wherein R₂₂ has the values listed in Table 14, were prepared using EDCI-HOBt coupling method described above for compound 99B, using an appropriate amino acid in place of Boc-β-alaine.

TABLE 14

| | | HPLC | |
|---|---|---|---|
| | | Retention | MS Data |
| Ex. | R₂₂ | Time (min) | (M + H)⁺ |
| 113 | 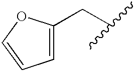 | 1.57^{a} | 594 |
| 114 | 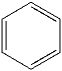 | 1.64^{a} | 604 |
| 115 | CF₃— | 1.66^{a} | 596 |
| 116 | CH₃CH₂— | 1.50^{a} | 556 |
| 117 | 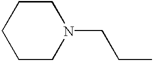 | 1.50 | 639 |
| 118 | (Me)₂NCH₂CH₂— | 1.42 | 599 |
| 119 | CH₃OCH₂— | 1.48 | 572 |
| 120 | 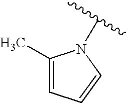 | 1.64 | 607 |
| 121 | 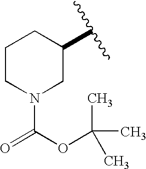 | 1.71 | 711 |
| 122 | 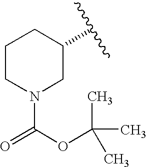 | 1.72 | 711 |

### MC-R Agonist Example 123

### 2-Amino-N-[1-(4-chloro-benzyl)-2-(4-cyclohexyl-4-[1,2,4]triazol-1-ylmethyl-piperidin-1-yl)-2-oxo-ethyl]-acetamide

### Step A

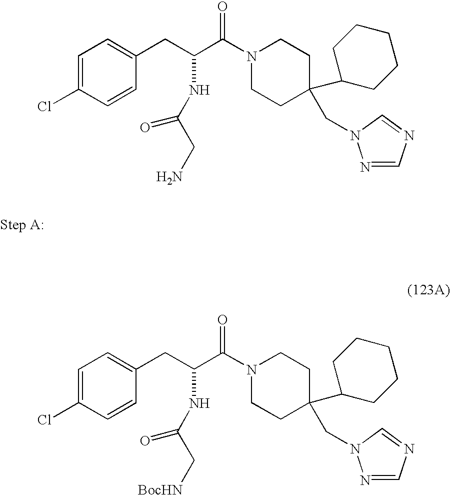

To a solution of compound 99A (83 mg, 0.19 mmol) and N-Boc-glycine (86 mg, 0.49 mmol) in DMF (2 mL) was added EDCl (93 mg, 0.49 mmol), HOBt (66 mg, 0.49 mmol) and DIPEA (135 μL, 0.78 mmol) at RT. The mixture was stirred at RT overnight and water (25 mL) was added. The aqueous layer was extracted with EtOAc (3×25 mL) and the combined organic layers were washed with a solution of sodium bicarbonate (25 mL), water (25 mL), brine (25 mL) dried (Na₂SO₄ anh.), filtered, and evaporated to afford the compound 123A which was used in the next step without purification.

### Step B

### EXAMPLE 123

To a solution of compound 123A (111 mg, 0.19 mmol) in DCM (5 mL) was added TFA (2.5 mL) at RT. The mixture was stirred at RT for 15 min. and evaporated under reduced pressure. The residue was purified using preparative HPLC and after evaporation, the residue was purified by automated solid phase extraction and concentrated in vacuo. The product was dissolved in a 4 M HCl solution in dioxane and lyophilized to yield 70 mg of Example 123 as the hydrochloride salt (66%). HPLC/MS (L), ret. time=1.41 min, purity 99%, MS pos. m/z 487 (M+H)⁺; HPLC/MS (B), ret. time=1.43 min, purity 97.8%, MS pos. m/z 487 (M+H)⁺; MS (Finigan TSQ 7000, ESI) m/z 487 (M+H)⁺; IR (νₘₐₓ, KBr) cm⁻¹ 3600-2854, 1683, 1625, 1456; ¹H NMR (400 MHz, MeOH-d₄) δ ppm (two rotamers; 1:1.2 ratio) 9.33 (1H, s), 9.26 (1H, s), 8.53 (1H, s), 8.46 (1H, s), 7.22-7.10 (4H, m), 4.99 (1H, t, J=8.0 Hz), 4.32 (2H, s, major rotamer), 4.30 (2H, s, minor rotamer), 3.68-3.50 (2H, m), 3.40-3.34 (1H, m), 3.27-3.21 (1H, m), 2.92-2.75 (2H, m), 1.75-0.76 (15H, m).

### MC-R Agonist Example 124

### 1-Methyl-azetidine-2-carboxylic acid[1-(4-chloro-benzyl)-2-(4-cyclohexyl-4-[1,2,4]triazol-1-ylmethyl-piperidin-1-yl)-2-oxo-ethyl]-amide

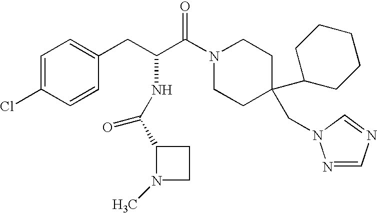

The procedure described for the preparation of Example 123 was used to prepare Example 124, using

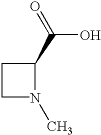

in place of N-Boc-glycine. Compound was prepared as the hydrochloride salt. HPLC/MS (ret. time)=1.55^{L} min; 1.84^{m} min; ¹H NMR. 400 MHz, MeOH-d₄) δ ppm (two rotamers, 1:2) 9.30 (m, 1H, broad), 8.52 (m, 1H, broad), 7.33 (d, 2H, J=8 Hz, major rotamer), 7.28 (d, 2H, J=8 Hz, minor rotamer), 7.24 (d, 2H, J=8 Hz, major rotamer), 7.22 (d, 2H, J=8 Hz, minor rotamer), 5.10 (m, 1H), 4.41 (s, 2H), 4.07-3.93 (m, 2H), 3.72-3.67 (m, 1H), 3.55-3.36 (m, 3H), 3.05-2.90 (m, 2H), 2.88 (s, 3H, major rotamer), 2.86 (s, 2H, minor rotamer), 2.77-2.65 (m, 1H), 2.40-2.19 (m,1H), 1.80 (m, 3H), 1.68 (m, 3H), 1.54-0.90 (11H, m).

### MC-R Agonist Example 125

### 1-Methyl-azetidine-2-carboxylic acid[1-(4-chloro-benzyl)-2-(4-cyclohexyl-4-[1,2,4]triazol-1-ylmethyl-piperidin-1-yl)-2-oxo-ethyl]-amide

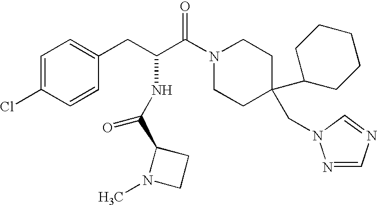

To a solution of 2-Amino-3-(4-chloro-phenyl)-1-(4-cyclohexyl-4-[1,2,4]triazol-1-ylmethyl-piperidin-1-yl)-propan-1-one (Compound 99A) (79 mg, 0.18 mmol) and (R)-1-methyl-azetidine-2-carboxylic acid

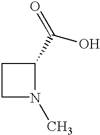

(32 mg, 0.28 mmol) in N,N-dimethylformamide (1.8 mL) was added 1-ethyl-3-(3-dimethylaminopropyl) carbodiimide (53 mg, 0.28 mmol), 1-hydroxybenzotriazole hydrate (37 mg, 0.28 mmol) and N,N-diisopropylethylamine (97 □L, 0.56 mmol) at rt. The mixture was stirred 12 hours and then the solution was purified using preparative HPLC (Column: Column S-5 Phenyl 20×100 mm. Acetonitrile-0.05% TFA/water: 7 min. gradient from 10% AcCN to 90% AcCN at 220 nm. Flow rate: 20 mL/min.) and collected fractions were concentrated in vacuo. A second purification using preparative HPLC was done (Column: Column X-Terra C-8 21.2×100 mm. Acetonitrile-5 mM NH₄OAc/water: 7 min. gradient from 10% AcCN to 90% AcCN at 220 nm. Flow rate: 20 ml/min) and collected fractions were concentrated in vacuo. The hydrochloride salt was made using a solution of 4 M HCl in dioxane and the salt was lyophilized to yield 30 mg of Example 110. (31%). HPLC/MS (Column: Xterra-C8 4.6×30 mm; Flow rate: 4 mL min, Solvent system: 0-100% B in 2 min. Solvent A: 10% CH₃CN-90 % H₂O-5 mM NH₄OAc; Solvent B: 90% CH₃CN-10% H₂O-5 mM NH₄OAc; UV: 220 nm; Micromass ZMD 2000, ESI): retention time 1.55 min, purity 92.4%, MS pos. m/z 527 (M+H)⁺; HPLC/MS (Column: YMC-Pack S5 Phenyl 4.6×50 mm; Flow rate: 3 mL/min, Solvent system: 0-100% B in 2 min. Solvent A: 10% CH₃CN-90% H₂O-0.05% TFA; Solvent B: 90% CH₃CN-10% H₂O-0.05% TFA; UV: 220 nm; Micromass ZMD 2000, ESI): retention time 1.83 min, purity 97.5%, MS pos. m/z 527 (M+H)⁺; MS (Finigan TSQ 7000, ESI) m/z 527 (M+H)⁺; HRMS calculated for C₂₈H₃₉ClN₆O₂ (M +H⁺)=527.290128; Found=527.291621; ¹H nmr (400 MHz, MeOH-d₄) ≢ ppm (two rotamers, 1:2) 9.60 (s, 1H, broad, minor rotamer), 9.57 (s, 1H, broad, major rotamer), 8.81 (dd, 1H, J=4, 8 Hz), 8.74 (s, 1H, broad, minor rotamer), 8.69 (s, 1H, broad, major rotamer), 7.34-7.23 (m, 4H), 5.08 (m, 1H), 4.46 (s, 2H, major rotamer), 4.44 (s, 2H, minor rotamer), 4.16-3.97 (m, 2H), 3.77-3.60 (m, 2H), 3.52-3.46 (m, 1H), 3.40-3.35 (m, 1H), 2.99 (d, 1H, J=8 Hz), 2.89 (s, 3H, major rotamer), 2.85 (s, 3H, minor rotamer), 2.80-2.71 (m, 1H), 2.54-2.45 (m, 1H), 1.80 (m, 3H), 1.68 (m, 3H), 1.54-0.90 (m, 11H).

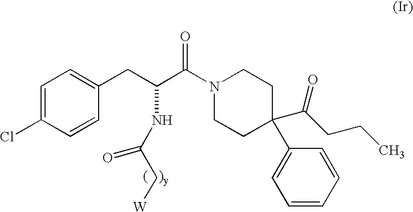

Compounds of formula (Ir), above, wherein the integer y and group W have the values listed in Table 15, were prepared following the same or similar procedure described above for Example 2, using a different amino acid in place of N-Boc-L-histidine in Step A.

TABLE 15

| | | | | HPLC | |
|---|---|---|---|---|---|
| | | | Purity | RT | Mass |
| Ex. No. | y | W | (%) | (min) | (M + H) |
| 126 | 2 | 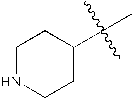 | 96 | 2.79 | 548.34 |
| 127 | 4 | 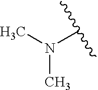 | 95 | 2.74 | 536.36 |

We claim:

1. A method of regulating cyclic adenoise 3′,5′ monophosphate (cAMP) production in a mammal comprising administering to the mammal a combination of (i) an amount of at least one compound effective for agonizing a melanocortin-receptor selected from MC-1R and MC-4R, and (ii) an amount of at least one compound effective for inhibiting cAMP phosphodiesterase (PDE).

2. The method according to claim 1, in which at least one of the amount of the melanocortin-receptor agonist and the amount of the cAMP-PDE inhibitor is a subtherapeutically-effective amount for treating a cAMP-associated disease, wherein administration of the combination provides a therapeutically-effective modulation of cAMP production for treating the cAMP-associated disease.

3. The method according to claim 2, in which the cAMP-associated disease is selected from at least one of inflammatory bowel disease, irritable bowel syndrome, rheumatoid arthritis, osteoarthritis, pancreatis, psoriasis, migraine, Alzheimer's Disease, Parkinson's disease, transplant rejection, asthma, acute respiratory distress syndrome, chronic obstructive pulmonary disease, stroke, ischemic brain disease, neurodegeneration resulting from stroke or ischemic brain disease, and the neurodegeneration of, and consequences of, traumatic brain injury.

4. The method according to claim 1, in which the melanocortin-receptor agonist is a highly selective MC-1R agonist.

5. The method according to claim 1, in which the melanocortin-receptor agonist is a highly selective MC-4R agonist.

6. The method according to claim 1, in which the at least one cAMP-PDE inhibitor is a PDE 3, 4, 7 and/or 8 inhibitor.

7. The method according to claim 1, in which the at least one cAMP-PDE inhibitor is a PDE 4 inhibitor.

8. The method according to claim 7, in which the at least one PDE 4 inhibitor is rolipram or ariflo.

9. The method according to claim 1, in which the at least one cAMP-PDE inhibitor is selected from theophylline, denbutyline, XT-44, roflumilast, revizinone, pimobendan, olprinone, cilomilast, piclamilast, hydroxynonyladenine, motapizone, and dipyridamole.

10. The method of claim 1, in which the at least one melanocortin-receptor agonist is selected from a compound having the formula (I), 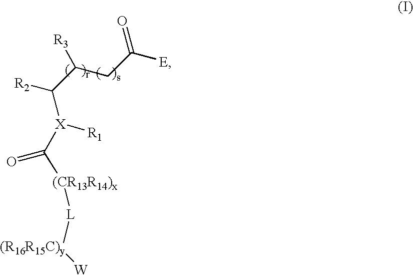
and a pharmaceutically-acceptable salt, hydrate, or prodrug thereof, in which:
L is a bond or —CH(G)-;
X is N or CH;
R₁ is hydrogen or C₁₋₆alkyl or is taken together with R₂ or R₃ to form a monocyclic or bicyclic aryl, cycloalkyl, heteroaryl or heterocycle;
R₂ is hydrogen, aryl, cycloalkyl, heteroaryl, or heterocyclo; or C₁₋₆alkyl or C₂₋₆alkenyl optionally substituted with one to three of hydroxy, alkoxy, halogen, cyano, trifluoromethyl, nitro, amino, alkylamino, aryl, cycloalkyl, heteroaryl, and/or heterocyclo; or R₂ is taken together with R₁ or R₃ to form a monocyclic or bicyclic aryl, cycloalkyl, heteroaryl or heterocycle;
R₃ is hydrogen or C₁₋₆alkyl or is taken together with R₁ or R₂ to form a monocyclic or bicyclic aryl, cycloalkyl, heteroaryl or heterocycle;
E is E₁, E₂, E₃ or E₄, wherein 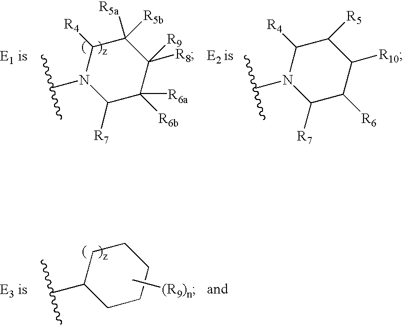
E₄ is —NR₁₁R₁₂;
G is selected from C₂₋₆alkenyl, A₃-aryl, —OR₁₈, A₁-heteroaryl, A₁-cyano, A₂ —OR₁₇, A₁-C(═O)R₁₈, A₁-CO₂R₁₈, A₁-C(═O)NR₁₈R₁₉, A₁-OC(═O)R₁₈, A₁-NR₁₈C(═O)R₁₉, A₁-OC(═O)NR₁₈R₁₉, A₁-NR₁₈CO₂R₁₉, A₁-NR₁₈SO₂R₁₇, A₁-SO₂R₁₇, A₁-NR₂₀C(═O)NR₁₈R₁₉, A₁-SR₁₈, A₁-heterocyclo, wherein A₁ is a bond, C₁₋₆alkylene or C₂₋₆alkenylene (straight or branched chain), A₂ is C₁₋₆alkylene or C₂₋₆alkenylene, and A₃ is C₂₋₆alkenylene;
W is selected from —NR₂₁R₂₂, —OR ₂₃, —NR₂₁C(═O)R₂₄, —NR₂₁CO₂R₂₄, amidino, guanidino, or a substituted or unsubstituted heterocyclo, heteroaryl, or cycloalkyl selected from azepinyl, azetidinyl, imidazolyl, imidazolidinyl, pyrazolyl, pyridyl, pyrazinyl, pyridazinyl, 1,2-dihydropyridazinyl, pyranyl, tetrahydropyranyl, piperazinyl, homopiperazinyl, pyrrolyl, pyrrolidinyl, piperidinyl, thiazolyl, tetrahydrothiazolyl, thienyl, furyl, tetrahydrofuryl, morpholinyl, isoquinolinyl, tetrahydroisoquinolinyl, tetrazolyl, oxazolyl, tetrahydro-oxazolyl, and C₃₋₇cycloalkyl, wherein said heteroaryl, heterocyclo or cycloalkyl groups may additionally have joined thereto an optionally substituted five-to-seven membered heterocyclic, heteroaryl, or carbocyclic ring;
R₄ and R₇ are independently selected from hydrogen, alkyl, substituted alkyl, halogen, hydroxy, alkoxy, and keto;
R₅, R₅ₐ, R_{5b}, R₆, R₆ₐ, R_{6b}, R₈ and R₉ are independently hydrogen, halogen, cyano, alkyl, substituted alkyl, alkenyl, alkynyl, cycloalkyl, heterocyclo, aryl, heteroaryl, —OR₂₅, —NR₂₅R₂₆, —SR₂₅—S(O)ₚR₂₆, —C(═O)R₂₅, —OC(═O)R₂₅, —CO₂R₂₅, —C(═O)N R₂₅R₂₆, —NR₂₅C(═O)R₂₆, —OC(═O)NR₂₅R₂₆, —NR₂₅CO₂R₂₆, —NR₂₇C(═O)NR₂₅R₂₆ or —NR₂₅SO₂R₂₆; or R₅ₐ, and R_{5b}, R₆ₐ and R_{6b}, or R₈ and R₉ taken together form a keto group (═O) or a monocyclic or bicyclic cycloalkyl or heterocyclo joined in a spiro fashion to ring E, or alternatively, R₅ₐ and/or R_{5b} together with R₈ and/or R₉, or R₆ₐ and/or R_{6b} together with R₈ and/or R₉, are taken to form a fused carbocyclic, heterocyclic, or heteroaryl ring;
R₁₀ is selected from hydrogen, alkyl, substituted alkyl, cycloalkyl, aryl, heteroaryl, and hetereocyclo;
R₁₁ is hydrogen or C₁₋₈alkyl;
R₁₂ is C₁₋₈alkyl, substituted C₁₋₁₈alkyl, or cycloalkyl;
R₁₃, R₁₄, R₁₅ and R₁₆ are selected independently of each other from hydrogen, alkyl, substituted alkyl, amino, alkylamino, hydroxy, alkoxy, aryl, cycloalkyl, heteroaryl, or heterocyclo, or R₁₃ and R₁₄, or R₁₅ and R₁₆, when attached to the same carbon atom, may join to form a spirocycloalkyl ring;
R₁₇ is alkyl, substituted alkyl, cycloalkyl, aryl, heterocyclo, or heteroaryl;
R₁₈, R₁₉, and R₂₀ are independently selected from hydrogen, alkyl, substituted alkyl, alkenyl, substituted alkenyl, ary , heteroaryl, cycloalkyl, heterocyclo, or C(═O)R₂₈; or when G is NH(C═O)R₁₉, R₁₉ may be a bond joined to W to define a heterocyclo ring;
R₂₁ and R₂₂ are selected from hydrogen, alkyl, and substituted alkyl;
R₂₃ and R₂₄ are independently selected from hydrogen, alkyl, substituted alkyl, aryl, heteroaryl, heterocyclo, and cycloalkyl;
R₂₅, R₂₆ and R₂₇ are independently selected from hydrogen, alkyl, substituted alkyl, cycloalkyl, aryl, heterocyclo, and heteroaryl; or R₂₅ and R₂₆ may join together to form a heterocyclo or heteroaryl, except R₂₆ is not hydrogen when joined to a sulfonyl group as in —S(O)ₚR₂₆ or —NR₂₅SO₂R₂₆;
R₂₈ is hydrogen, alkyl, or substituted alkyl;
n is 0, 1, 2, 3 or 4;
p is 1, 2, or 3;
r and s are 0 or 1;
x is 0, 1, or 2;
y is 0, 1, 2, 3 or 4; and
z is 0, 1, or 2.

11. The method according to claim 10, in which the melanocortin receptor agonist is a compound having the formula, 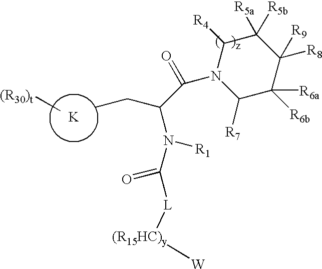
or a pharmaceutically-acceptable salt, hydrate, or prodrug thereof, in which:
R₁ is hydrogen or C₁₄alkyl;
R₁₅ is hydrogen, C₁₋₄alkyl, or substituted C₁₋₄alkyl,
K is aryl or heteroaryl;
R₃₀ is C₁₋₄alkyl, hydroxy, methoxy, ethoxy, halogen, nitro, cyano, amino, C₁₋₄alkylamino, phenyl, or C(═O)phenyl;
t is 0, 1, or 2; and
z is 0 or 1.

12. A pharmaceutical composition comprising a combination of (i) an amount of at least one compound effective for agonizing a melanocortin-receptor selected from MC-1R and MC-4R in a first pharmaceutically-acceptable carrier or diluent, and (ii) an amount of at least one compound effective for inhibiting cAM-PDE in a second pharmaceutically-acceptable carrier or diluent.

13. The pharmaceutical composition according to claim 12, in which the at least one melanocortin-receptor agonist and the at least one compound cAMP-PDE inhibitor is contained in a single-dosage unit.
